(19) United States
(12) Patent Application Publication
(10) Pub. No.: US 2025/0157653 A1
(43) Pub. Date: May 15, 2025

(54) MEDICAL DATA SUMMARY INTERFACE SYSTEM

(71) Applicant: GE Precision Healthcare LLC, Waukesha, WI (US)

(72) Inventors: Marine Loth, Noisy le Roi (FR); Romane Amice, Paris (FR); Cedric Castanier, Yvelines (FR); Marwa Guennour, Yvelines (FR); Louise Lelievre, Clamart (FR); Sherazade Aknoun, Verrières-Le-Buisson (FR)

(21) Appl. No.: 18/931,945

(22) Filed: Oct. 30, 2024

(30) Foreign Application Priority Data
Nov. 17, 2023 (FR) 2312647

(51) Int. Cl. G16H 50/20; G16H 10/60; G16H 30/20

(52) U.S. Cl. CPC G16H 50/20; G16H 10/60; G16H 30/20

(57) ABSTRACT

Various systems and methods are presented regarding presentation of patient medical data to enable expedited review and/or detailed analysis of the patient medical data. A summary screen can be configured to present a summary of the plethora of available data, wherein the summary screen presents data identified to enable expeditious diagnosis of a patient's condition, while more detailed information is available for presentation on a pop-up screen(s) or navigation to a specific application configured to present information at a greater level of detail/granularity. The summary screen can be configured to present one or more interactive images (e.g., an MRI) in conjunction with text providing further description of the diagnosis, medical data, and suchlike.

### CROSS-REFERENCE TO RELATED APPLICATIONS

This application claims priority to French Provisional Patent Application No. 2312647 filed on Nov. 13, 2023, entitled “A MEDICAL DATA SUMMARY INTERFACE SYSTEM”. The entireties of the aforementioned application are incorporated by reference herein.

### TECHNICAL FIELD

This application relates to systems and techniques facilitating analysis and presentation of medical information regarding a patient.

### BACKGROUND

Time available to review a medical imaging exam tends to become shorter as the workload and working requirements of medical staff increases (e.g., as radiologists and clinicians work intensifies). However, with continued application of artificial intelligence (AI) and machine learning (IL) systems and technologies, an increased number of tasks relating to medical imaging and medical condition diagnosis are being automated.

### SUMMARY

The following presents a simplified summary of the disclosed subject matter to provide a basic understanding of one or more of the various embodiments described herein. This summary is not an extensive overview of the various embodiments. It is intended neither to identify key or critical elements of the various embodiments nor to delineate the scope of the various embodiments. The sole purpose of the Summary is to present some concepts of the disclosure in a streamlined form as a prelude to the more detailed description that is presented later.

According to one or more embodiments, a system is presented, wherein the system comprises at least one processor and at least one memory coupled to the at least one processor and having instructions stored thereon, wherein the system can be configured to automatically and dynamically generate one or more summaries of patient information, further identify a potential medical condition for the patient, and further provide a potential diagnosis of the medical condition for subsequent review, in accordance with an embodiment. In response to the at least one processor executing the instructions, the instructions facilitate performance of operations, comprising vectoring content of a patient's medical information to generate a first vectored content, and further identifying, based on the first vectored content, second vectored content in a digital library comprising medical condition information, wherein a medical condition defined by the second vectored content is assigned to the patient based on threshold similarity between the first vectored content and the second vectored content. In a further embodiment, the operations can further comprise generating a summary of the medical condition of the patient, wherein the summary is generated by a computer-implemented language model operating on at least one of the first vectored content or the second vectored content, and further presenting the summary to facilitate subsequent investigation of the patient's medical condition.

In an embodiment, the summary can include a hyperlinked statement, wherein the hyperlinked statement is a digital reference to information in the patient's medical information from which the hyperlinked statement was generated, wherein the operations can further comprise detecting selection of the hyperlinked statement, and further presenting the patient's medical information from which the hyperlinked statement was derived.

In an embodiment, the patient's medical information can comprise at least one of personal data pertaining to the patient, medical data pertaining to the patient, an image of the patient, or an image of at least one organ of the patient.

In an embodiment, the operations can further comprise determining, from the patient's medical information, a diagnosis of the patient's medical condition, wherein the diagnosis comprises generating third vectored content from the patient's medical information and identifying a fourth vectored content in the digital library comprising the medical condition information being similar to the third vectored content, and further presenting the diagnosis for further investigation of the patient's medical condition.

In another embodiment, the operations can further comprise determining a degree of confidence in the appropriateness of the diagnosis to the patient's medical condition, wherein the degree of confidence is based on a measure of similarity between the third vectored content and the fourth vectored content, and further presenting the degree of confidence with the diagnosis.

In a further embodiment, the operations can further comprise receiving a confirmation of the diagnosis, and in response to receiving the confirmation of the diagnosis, the operations can further comprise generating a report presenting a summary of the patient's medical condition and the diagnosis.

In an embodiment, the patient's medical information is first medical information and the summary of the medical information is a summary of the first medical information, wherein the operations can further comprise detecting an edit applied to the summary of the first medical information, wherein the edit comprises addition or removal of information from the summary of the first medical information.

In another embodiment, the operations can further comprise updating the first medical information to generate second medical information, wherein the second medical information comprises the edit applied to the summary of the first medical information.

In further embodiments, a computer-implemented method is provided, wherein the method comprises generating, by a device comprising at least one processor, a summary, wherein the summary summarizes two or more items of medical information pertaining to a patient, wherein the summary is generated by a large language model operating on the two or more items of medical information pertaining to the patient, and further identifying, by the device, an image associated with the patient and pertains to content of the summary, wherein the image is included in the two or more items of medical information pertaining to the patient. In a further embodiment, the computer-implemented method can further comprise generating, by the device, a diagnosis of a medical condition of the patient, wherein the diagnosis is generated based in part on similarity between information presented in the summary and at least one diagnosis present in a digital library comprising medical diagnosis information, wherein the similarity is assessed based on a first vectored content generated from the information presented in the summary and a second vectored content generated from the at least one diagnosis in the digital library comprising medical diagnosis information. In a further embodiment, the computer-implemented method can further comprise presenting, by the device, the summary in conjunction with at least one of the diagnosis or the image.

In another embodiment, the summary can include a hyperlink to original information sourced from the two or more items of medical information from which the summary was generated, wherein the computer-implemented method further comprises detecting, by the device, selection of the hyperlink and further, in response to detecting, by the device, selection of the hyperlink, presenting, by the device, the original information in conjunction with the summary in conjunction with at least one of the diagnosis or the image.

In an embodiment, the hyperlink in the summary is a first hyperlink to first information sourced from the two or more items of medical information which the summary was generated, and wherein the medical image further comprises a second hyperlink pertaining to second information available in the two or more items of medical information,

In a further embodiment, the computer-implemented method can further comprise detecting, by the device, an edit to the summary, and further, updating, by the device, information in the original information in accordance with the edit to the summary.

In another embodiment, the edit to the summary comprises addition, modification, or removal of information from the summary.

In a further embodiment, the computer-implemented method can further comprise updating, by the device, the original information to generate second information, wherein the second information comprises the edit applied to the summary of the original information.

In another embodiment, the original information comprises at least one of a medical image or medical condition information.

In a further embodiment, the computer-implemented method can further comprise generating, by the device, a degree of confidence in the applicability of the diagnosis to the patient medical condition, wherein the degree of confidence is based on a measure of similarity between the first vectored content generated from the information presented in the summary and the second vectored content generated from the at least one diagnosis in the digital library comprising medical diagnosis information. In a further embodiment, the computer-implemented method can further comprise presenting, by the device, the degree of confidence in conjunction with the diagnosis.

Further embodiments can include a computer program product stored on a non-transitory computer-readable medium and comprising machine-executable instructions, wherein, in response to being executed, the machine-executable instructions cause a system to perform operations, comprising receiving selection of a hyperlink, wherein the hyperlink is included in a summary of original patient information pertaining to a patient, wherein the summary is generated by a computer-implemented model configured to summarize content in the original patient information pertaining to the patient, and the hyperlink is a digital reference to original information in original patient information pertaining to the patient from which the hyperlinked statement was generated. The operations can further comprise identifying the original information in the original patient information digitally referenced by the hyperlink, and further presenting the original information in conjunction with the summary.

In an embodiment, the hyperlink is a first hyperlink, wherein the original patient information pertaining to the patient is first patient information utilized to create the summary. In a further embodiment, the operations can further comprise receiving selection of a second hyperlink, wherein the second hyperlink is included in an image presented in conjunction with the summary, and the second hyperlink links to second patient information pertaining to the patient, further identifying the second patient information pertaining to the hyperlink, and further presenting the second patient information.

In another embodiment, the operations can further comprise comparing the first patient information and the second patient information with medical condition information, wherein the medical condition information is stored in a digital library comprising medical diagnosis information and includes information pertaining to a medical condition. In a further embodiment, the operations can further comprise identifying, in the medical condition information, the medical condition, wherein identification of the medical condition information comprises comparing similarity of at least one of first vectored content generated from the first patient information or second vectored content generated from the second patient information with third vectored content generated from the medical condition information stored in the digital library comprising medical diagnosis information. In another embodiment, the operations can further comprise generating a diagnosis of the medical condition, wherein the diagnosis is generated based on a comparison of at least one of the first patient information or the second patient information with diagnosis information included in the digital library comprising medical diagnosis information, wherein the diagnosis is based on measure of similarity between at least one of the first vectored content or the second vectored content with a third vectored content generated for the diagnosis, and further presenting the diagnosis for review.

In another embodiment, the operations can further comprise generating a degree of confidence for the diagnosis, wherein the degree of confidence is based on at least one of a first relatedness measure of the first vectored content to the medical condition or a second relatedness measure of the second vectored content to the medical condition, and further presenting the degree of confidence in conjunction with the diagnosis.

### DESCRIPTION OF THE DRAWINGS

One or more embodiments are described below in the Detailed Description section with reference to the following drawings.

FIG. 1 illustrates a system which can be utilized to control presentation of information and images, according to at least one embodiment.

FIG. 2 illustrates a patient selection screen, according to an embodiment.

FIG. 3 illustrates a summary screen, according to an embodiment.

FIG. 4 presents a summary screen, according to an embodiment.

FIG. 5 presents a summary screen, according to an embodiment.

FIG. 6 presents a summary screen, according to an embodiment.

FIG. 7 illustrates sub-components included in a condition component, in accordance with one or more embodiments.

FIG. 8 presents a schematic illustrating operational flow for review of a patient's medical condition, in accordance with one or more embodiments.

FIG. 9 presents an example computer-implemented method for generating/presenting a summary of medical information for review, in accordance with an embodiment.

FIG. 10 presents an example computer-implemented method for automatically and dynamically generating one or more summaries of patient information, further identify a potential medical condition for the patient, and further providing a potential diagnosis of the medical condition for subsequent review, in accordance with an embodiment.

FIG. 11 presents an example computer-implemented method for automatically and dynamically generating one or more summaries of patient information, further identify a potential medical condition for the patient, and further providing a potential diagnosis of the medical condition for subsequent review, in accordance with an embodiment.

FIG. 12 presents an example computer-implemented method for automatically and dynamically generating one or more summaries of patient information, further identify a potential medical condition for the patient, and further providing a potential diagnosis of the medical condition for subsequent review, in accordance with an embodiment.

FIG. 13 is a block diagram illustrating an example computing environment in which the various embodiments described herein can be implemented.

FIG. 14 is a block diagram illustrating an example computing environment with which the disclosed subject matter can interact, in accordance with an embodiment

### DETAILED DESCRIPTION

The following detailed description is merely illustrative and is not intended to limit embodiments and/or application or uses of embodiments. Furthermore, there is no intention to be bound by any expressed and/or implied information presented in any of the preceding Background section and/or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

It is to be understood that when an element is referred to as being “coupled” to another element, it can describe one or more different types of coupling including, but not limited to, chemical coupling, communicative coupling, electrical coupling, electromagnetic coupling, operative coupling, optical coupling, physical coupling, thermal coupling, and/or another type of coupling. Likewise, it is to be understood that when an element is referred to as being “connected” to another element, it can describe one or more different types of connecting including, but not limited to, electrical connecting, electromagnetic connecting, operative connecting, optical connecting, physical connecting, thermal connecting, and/or another type of connecting.

As used herein, “data” can comprise metadata. Further, ranges A-n are utilized herein to indicate a respective plurality of devices, components, signals etc., where n is any positive integer.

Rather than presenting a plethora of information regarding a patient's medical condition and accordingly, requiring a medical expert to navigate through the wealth of data (e.g., including images), advantage can be taken of AI/ML technologies to identify/determine insights (aka findings, potential diagnoses, and suchlike), summarise the information, and further, present the information as one or more summary screens presented on a summary interface. In an embodiment, one or more images can be presented regarding one or more findings pertaining to a patient's condition, wherein the one or more images can be accompanied by a summary of findings. As well as presenting summarized information, the summary screen(s) can be configured to present a higher level of information, e.g., as required to enable medical diagnosis of a patient. Further, the summary screen(s) can include one or more medical images (e.g., a MRI view) as well as a textual summary of information pertinent to the patient's medical condition. The medical image(s) and summary text can include various hyperlinks, that upon selection, can cause a pop-up window to be presented disclosing further information, such as dedicated views focused on a region of interest (e.g., a targeted finding), wherein the information can be presented in a manner determined to be relevant for further investigation, such as usable orientation and view type. Further, interaction with the medical images enables rotation, zooming, panning, etc. Hence, medical personnel can initially be presented with the summary screen(s), and, in conjunction with respective selection of text and image manipulation, navigation of the information enables review at the summary level through to drilling down to detailed information and/or replacement of the summarized information with a traditional screen presenting the plethora of data as well as access/present a dedicated application with which the medic can interact with further. Hence, while there may be a wealth of data regarding a patient's condition available for presentment to a medical expert, advantage can be taken of the application of AI/ML technologies to enable a medical specialist to expeditiously reach a diagnosis, and further, reduce a degree of unnecessary interaction of the medical expert with the patient data.

An advantage of the one or more systems, computer-implemented methods and/or computer program products presented herein can be enabling presentation of a summary of patient data enabling a medical professional to expeditiously access information to enable efficient and quick diagnosis of the patient's condition, while minimizing the amount of time the medical professional has to engage with the data to make the diagnosis. Accordingly, advantage can be taken of AI/ML technology to parse/summarize the patient data and limit an initial presentation of patient data to information that is pertinent to the diagnosis, wherein the initial presentation of patient data can comprise of one or more findings, which upon initial or subsequent review can be utilized to assist with determination of one or more diagnoses.

It is to be appreciated that while the various embodiments presented herein are directed towards application of AI/ML to summarizing and presenting medical data, the embodiments are equally applicable to any comparable use, e.g., presentation of veterinary information, presentation of financial data, failure diagnostics, and suchlike.

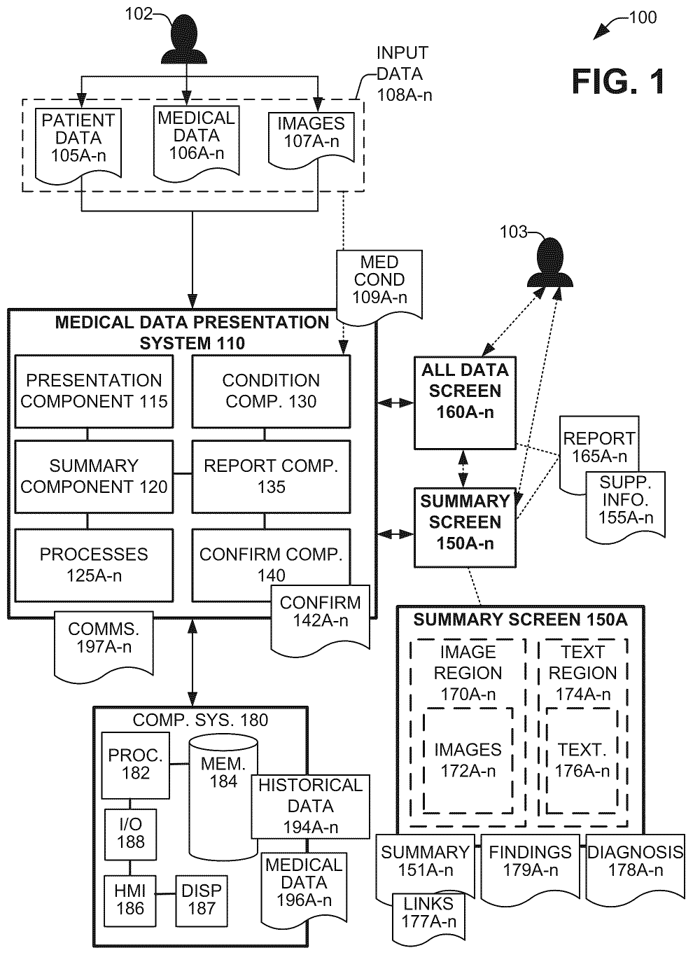

Turning now to the drawings, FIG. 1 illustrates a system 100 that can be utilized to control presentation of information and images, according to at least one embodiment.

System 100 comprises a medical data presentation system (MDPS) 110. As shown, MDPS 110 can be configured to receive information and data regarding a condition (e.g., a medical condition, a physical condition, a mental condition, and the like) of a patient 102, wherein the information can include patient data 105A-n, medical data 106A-n, and/or medical images 107A-n. For the sake of readability, patient data 105A-n, medical data 106A-n, and/or images 107A-n are combined in FIG. 1 as input data 108A-n. It is to be appreciated that while the term input data 108A-n is being used, any of the patient data 105A-n, medical data 106A-n, and/or images 107A-n may be being discussed. Further, the terms patient data 105A-n, medical data 106A-n, images 107A-n, and/or input data 108A-n can be used interchangeably. Hence, while a specific embodiment mentions images 107A-n, the embodiment can equally pertain to any of patient data 105A-n, medical data 106A-n, and/or input data 108A-n.

Patient data 105A-n can include name, address, contact information, date of birth, gender, identifier (e.g., social security number, national health service identification, etc.), and suchlike, pertaining to patient 102. Medical data 106A-n can include medical condition data regarding the patient 102, medical history, medication history, heart rate, blood levels, and suchlike. Images 107A-n can include any images associated with treatment of patient 102, e.g., magnetic resonance imaging (MRI), X-ray image, mammogram image, scans, and suchlike. Any of patient data 105A-n, medical data 106A-n, images 107A-n, and/or input data 108A-n can be sourced from any available resource (e.g., an electronic medical records (EMR) system, scanned documents, a medical imaging system, physical entry via a human machine interface (HMI) 186, and the like).

MDPS 110 can further include a presentation component 115 configured to control presentation on any of data 108A-n on a display (e.g., display 187). In an embodiment, presentation component 115 can be configured to control whether data 108A-n is presented in a largely complete manner, e.g., the patient data 105A-n, medical data 106A-n, and/or images 107A-n are presented in their entirety on one or more all data screens 160A-n or in summary form, e.g., as one or more summaries 151A-n on summary screen(s) 150A-n. Presentation of a totality of input data 108A-n on all data screens 160A-n is typical of a conventional system/approach, however, the volume of data can render the task of medical condition diagnosis by a caregiver 103 to be time consuming and also potentially fraught with misdiagnosis given the wealth of data to review. However, by utilizing the one or more embodiments presented herein regarding identification and summary presentation of data enables caregiver 103 to quickly, accurately, and with confidence, make a determination of whether a medical condition exists or not. The term caregiver 103 is used herein to represent any person involved in reviewing medical data, one or more findings, and making a diagnosis based thereon, such as a doctor, nurse, radiologist, oncologist, medical specialist, medical professional, and the like.

In another embodiment, the presentation component 115 can be configured to utilize summary screens 150A-n to present one or more portions of the entirety of information available for patient 102.

MDPS 110 can further include a summary component 120, wherein, in an embodiment, the summary component 120 can be configured to control presentation/depiction of patient data 105A-n, medical data 106A-n, images 107A-n, and suchlike, e.g., as one or more summaries 151A-n on summary screens 150A-n. Summary screens 150A-n can respectively comprise one or more image regions 170A-n configured to include one or more images 172A-n. in the one or more summaries 151A-n.

Images 172A-n can include images 107A-n pertaining to patient 102 (e.g., as part of input data 108A-n), as well as images identified and extracted from historical data 194A-n/medical data 196A-n.

Further, summary screens 150A-n can respectively comprise one or more text regions 174A-n configured to include one or more text 176A-n. In an embodiment, text 176A-n can be included in the one or more summaries 151A-n. In an embodiment, text 176A-n can include one or more findings 179A-n, wherein the findings 179A-n can be automatically generated by the condition component 130. Text 176A-n can include text, alphanumerics, values, symbols, etc., in input data 108A-n pertaining to patient 102, as well as text/information identified and extracted from historical data 194A-n/medical data 196A-n. Per the various embodiments presented herein, the terms medical condition 109A-n and findings 179A-n can be used interchangeably or in isolation. For example, caregiver 103 can be aware of a medical condition 109A of patient 102 (per the line from input data 108A-n to MDPS 110, where medical condition 109A can be considered to be included in input data 108A-n) and a finding 179A can be determined that confirms the medical condition 109A. In another example, caregiver 103 is unaware of a medical condition of patient 102, a finding 179B is generated/determined by MDPS 110 (and one or more included components), from which caregiver 103 derives a medical condition 109B. In another example, caregiver 103 is unaware of a medical condition of patient 102, a finding 179C is generated/determined by MDPS 110 (and one or more included components), from which MDPS 110 (and one or more included components) derives a medical condition 109C, and further, MDPS 110 (and one or more included components) derives a potential diagnosis 178C of the medical condition 109C (as further described).

Summary screens 150A-n can further include one or more potential diagnoses 178A-n automatically generated by condition component 130, wherein the potential diagnoses 178A-n can be subsequently accepted/rejected by caregiver 103. It is to be appreciated that the terms diagnosis/diagnoses 178A-n and findings 179A-n are used interchangeably herein, indicating that a finding 179A-n can become a diagnosis 178A-n, and further, a diagnosis 178A-n can be derived from a finding 179A-n.

Further, as shown in FIG. 1, supplemental information 155A-n can be present, whereby supplemental information 155A-n can comprise available text, information, images that while not currently being presented on a respective summary screen 150A-n is available for presentment. For example, while first text 176A is presented on summary screen 150A, with first text 176A being a summary 151A, first text 176A can include one or more links 177A-n (a.k.a. hyperlinks, e.g., a digital reference to data, presented with first information/data that when clicked, presents second information/data) which when selected can cause one or more portions (e.g., images, text) of the supplemental information 155A-n to presented on summary screen 150A-n (e.g., as a pop-up). In an embodiment, presentation component 115 can be configured to determine whether data 108A-n is to be presented on an all data screen 160A-n or a summary screen 150A-n, and summary component 120 can be configured to control how a summary of data 108A-n is presented on a summary screen 150A-n. In an embodiment, links 177A-n can also link to respective processes 125A-n, to facilitate implementation of a process 125A-n configured to assist in review/analysis of data 108A-n, images 172A-n, text 176A-n, findings 179A-n, diagnoses 178A-n, etc., as further described.

In another embodiment, the summary component 120 can be further configured to identify respective images 172A-n to present on the summary screens 150A-n. For example, while numerous images 172A-n may be available for presentment on a summary screen 150A-n, the summary component 120 can be configured to identify an image 172A-n which enables the caregiver 103 to make an accurate/expeditious decision (e.g., a diagnosis 178A-n) regarding a medical condition 109A-n of patient 102. For example, as further described, a condition component 130 can determine a finding 179A-n and/or proposed diagnosis 178A-n. The summary component 120 can receive the finding 179A-n, for example, and based on the finding 179A-n, in conjunction with one or more processes 125A-n, the summary component 120 can identify an image 172A-n applicable to the finding 179A-n (e.g., enables caregiver 103 to readily discern the finding 179A-n), and present the identified image 172A-n on a summary screen 150A-n.

As further shown, MDPS 110 can further include a condition component 130 configured to automatically review any or all of input data 108A-n to determine/provide an indication, e.g., a finding 179A-n, of whether or not patient 102 has a health condition of concern. As further described, during generation of findings 179A-n and/or diagnosis 178A-n, data 108A-n can be supplemented by medical data 196A-n (e.g., in memory 184), wherein medical data 196A-n can comprise the corpus of available medical knowledge. Accordingly, as part of a diagnosis process, condition component 130 can be configured to compare features/criteria/variables/parameters/content present in data 108A-n with features/criteria/variables/parameters/content medical data 196A-n pertaining to the potential medical condition 109A-n of patient 102.

As further described, MDPS 110 can include a collection of processes 125A-n, wherein processes 125A-n can include respective AI and ML technologies and techniques. Processes 125A-n (aka an application) can be directed to the plethora of medical conditions 109A-n experienced by humanity (e.g., per medical data 196A-n), and accordingly, the medical conditions 109A-n that patient 102 may experience during their lifetime.

MDPS 110 can further include a report component 135 configured to generate a report 165A-n. In an embodiment, as further described, report 165A-n can be a structured document generated based on the information presented on the summary screen 150A-n, wherein the summary screen 150A-n can function as a viewport. Report 165A-n can be of any suitable format/filetype, such as a PDF, DICOM PDF, word processor document, spreadsheet, and suchlike. As further described, report 165A-n can be configured to be presented on summary screen 150A-n, printed by a printer (not shown) associated with MDPS 110, exported to a remote system (e.g., via I/O 188), and suchlike. In an embodiment, report 165A-n can function as a medical file regarding the medical condition 109A-n of patient 102 and further the respective procedures and review utilized by caregiver 103 to diagnose patient 102's medical condition 109A-n. Accordingly, report 165A-n can function as a digital file regarding patient 102. In an embodiment, the respective findings 179A-n generated by the condition component 130 can be presented in report 165A-n in conjunction with the diagnosis 178A-n confirmed by the caregiver 103, such a report format can be provided to a patient or other entity concerned with the diagnosis 178A-n of patient 102 and the data (e.g., findings 179A-n) leading to the diagnosis. In another embodiment, report 165A-n can present the diagnosis 178A-n derived by the caregiver 103 in conjunction with the respective one or more findings 179A-n and any interactions/edits/annotations made by the caregiver 103 when deriving the diagnosis 178A-n.

In an embodiment, MDPS 110 can include a confirmation component 140. To ensure compliance with respective health care standards, legal standards, etc., that govern the respective health care entities around the globe, the confirmation component 140 can be utilized to ensure that caregiver 103 has reviewed the respective information, findings 179A-n, any potential diagnosis 178A-n, etc., generated and presented by the MDPS 110 (e.g., via summary screen 150A-n or all data screen 160A-n) and has signed off (e.g., confirmation 142A-n) on the diagnosis 178A-n/medical condition 109A-n/finding 179A-n of patient 102, as represented by the information, diagnosis 178A-n, finding 179A-n, etc., as generated and presented by MDPS 110. In an example embodiment, caregiver 103 can sign in to MDPS 110 (e.g., with their respective credentials, password, etc.), review/interact with information presented on the summary screen 150A-n, and when caregiver 103 is satisfied/in agreement that MDPS 110 (and/or caregiver 103) has accurately identified and/or diagnosed the one or more medical conditions 109A-n of patient 102, caregiver 103 can sign off the information with confirmation 142A-n. In the event of the caregiver 103 signing off the information, etc., the report component 135 can be configured to generate report 165A-n.

In an example embodiment of implementation, the condition component 130 can be configured to analyze/review/process data 108A-n, and while initial review of data 108A-n may be focused on a first medical condition 109A, the review can also be configured to analyze for other conditions 109B-n that may not be initially of concern to caregiver 103. For example, patient 102 has been involved in an automobile accident and respective medical procedures (e.g., imaging) are being performed to determine if patient 102 is suffering from internal bleeding, whether their organs (e.g., liver, kidneys, and suchlike) have been damaged as a result of the accident, and suchlike. Hence, a first series of processes 125A-n are being employed by the condition component 130 to identify and assess degree of bodily injury resulting from the automobile accident, e.g., generating or more findings 179A-n relating a first patient condition 109A. During the assessment of the organs, an unexpected mass may be detected in the one or more images 107A-n. This can automatically trigger, at the condition component 130, implementation of a second series of processes 125A-n, wherein the second series of processes 125A-n are configured to determine presence of a cancerous growth/tumor, as presented in one or more findings 179A-n relating to the second patient condition 109B. Hence, while the patient 102 is initially being assessed for a first condition 109A, application of the collection of various processes 125A-n enables expeditious detection/determination of a second condition 109B, the presence of which was not initially anticipated by caregiver 103. The term “findings” (e.g., findings 179A-n) is used herein with reference to an observation, determination, discovery, and the like, regarding a medical condition of a patient, e.g., patient 102.

Generally, some 50-90% reviews performed by AI/ML of performed medical procedures indicate that patient 102 is not suffering a medical condition 109A-n of concern, e.g., a mammogram procedure indicates patient 102 does not have cancer. In a conventional approach, caregiver 103 may have to review a wealth of medical data for patient 102 (e.g., presented on an all data screen 160A-n), which can be time consuming. By utilizing one or more processes 125A-n that are directed towards the medical condition 109A-n of concern (e.g., cancer), information comprising medical data 106A-n and/or images 107A-n can be specifically targeted by the processes 125A-n. Accordingly, the pertinent information, e.g., one or more summaries 151A-n, images 172A-n and text 176A-n comprising findings 179A-n identified by processes 125A-n, and the like, can be presented on summary display 150A-n by the summary component 120. Given the focused nature of the one or more summaries 151A-n, images 172A-n, and/or text 176A-n, caregiver 103 can review the data presented on the summary display 150A, arrive at a diagnosis 178A-n, and sign off using the confirmation component 140, and report 165A-n can be generated. Per the embodiments presented herein, with application of the summary component 120, processes 125A-n, findings 179A-n, and utilization of summary display 150A-n, caregiver 103 can quickly make a determination that a medical condition 109A-n of interest does not exist. Hence, given the anticipated 50-90% of medical procedures generate negative results, caregiver 103 is able to spend a minimal amount of time reviewing patient 102's medical health, thereby freeing caregiver 103's time to attend to other issues, e.g., patient care issues, at a hospital, in an ambulance, at a general practitioner office, etc.

As shown, system 100 can further include historical data 194A-n, wherein historical data 194A-n can comprise previously utilized summary screens 150A-n, previously presented images 172A-n, previously presented text 176A-n, prior diagnosis' 178A-n, prior findings 179A-n, prior interaction by one or more caregivers 103A-n with medical data 196A-n, supplemental information 155A-n, information presented on the respective summary screens 150A-n, image regions 170A-n, images 172A-n, text regions 174A-n, text 176A-n, during generation of reports 165A-n, prior diagnosis confirmations 142A-n, previously generated reports 165A-n, and suchlike. Accordingly, processes 125A-n can be trained as a function of prior interactions with particular information by a caregiver 103A-n. Hence, when first text 176F was previously presented on summary screen 150A-n, if further information was sought by the caregiver 103A-n, the importance of the further information (e.g., medical data 106F) can be established such that after repeated/separate instances of interaction, a process 125F associated with first text 176F can be trained to present the medical data 106F in the textual summary region 174A-n. Further, by frequently training the respective processes 125A-n, as new research is obtained regarding a parameter/measure/criteria being determined by the medical community to be playing a greater/lesser role in diagnosing a condition 109A-n, the respective processes 125A-n can be trained to reflect the greater/lesser importance of a particular criteria, finding 179A-n, etc., in diagnosing a condition 109A-n, and whether information relating to the particular criteria should be included/removed during creation of the summary screen 150A-n by the summary component 120.

It is to be appreciated that the various processes 125A-n and operations presented herein are simply examples of respective AI and ML operations and techniques, and any suitable AI/ML model/technology/technique/architecture can be utilized in accordance with the various embodiments presented herein. In an aspect, processes 125A-n can operate singly or in combination to create one or more applications configured to be implemented regarding identifying a particular medical condition 109A-n, e.g., a collection of processes 125A-n forming an application to identify issues relating to patient 102's cardio health. Processes 125A-n can be based on application of terms, phrases, criteria, parameters, variables, and suchlike, in input data 108A-n, historical data 194A-n, medical data 196A-n, etc. Summary component 120 can be utilized to implement processes 125-n in conjunction with MDPS 110 and any components included in MDPS 110. An example process 125A-n can include a vectoring technique such as bag of words (BOW) text vectors, and further, any suitable vectoring technology can be utilized, e.g., Euclidean distance, cosine similarity, etc. Other suitable AI/ML technologies/processes 125A-n that can be applied include, in a non-limiting list, any of vector representation via term frequency-inverse document frequency (tf-idf) capturing term/token frequency in the input data 108A-n versus terms/tokens present in medical data 196A-n, historical data 194A-n, etc. Other applicable AI/ML technologies include, in a non-limiting list, neural network embedding, layer vector representation of terms/categories (e.g., common terms having different tense), bidirectional and auto-regressive transformer (BART) model architecture, a bidirectional encoder representation from transformers (BERT) model, a diffusion model, a variational autoencoder (VAE), a generative adversarial network (GAN), a language-based generative model such as a large language model (LLM), a generative pre-trained transformer (GPT), a long short-term memory (LSTM) network/operation, a sentence state LSTM (S-LSTM), a deep learning algorithm, a sequential neural network, a sequential neural network that enables persistent information, a recurrent neural network (RNN), a convolutional neural network (CNN), a neural network, capsule network, a machine learning algorithm, a natural language processing (NLP) technique, sentiment analysis, bidirectional LSTM (BiLSTM), stacked BiLSTM, and suchlike. Accordingly, in an embodiment, implementation of the summary component 120, presentation component 115, condition component 130, and suchlike, enables plain/natural language programming/annotation/correlation of the input data 108A-n with any of medical data 196A-n, historical data 194A-n, etc., to generate the summary images 172A-n and text 176A-n presented on summary screens 150A-n.

Language models, LSTMs, BARTs, etc., can be formed with a neural network that is highly complex, for example, comprising billions of weighted parameters. Training of the language models, etc., can be conducted, e.g., by presentation component 115, summary component 120, condition component 130, etc., with datasets, whereby the datasets can be formed using any suitable technology, such as data in historical data 194A-n, medical data 196A-n, input data 108A-n, and suchlike. Further, as previously mentioned, historical data 194, medical data 196, input data 108A-n, patient data 105A-n, medical data 106A-n, images 107A-n, text176A-n, images 172A-n, supplemental information 155A-n, summaries 151A-n, and suchlike, can comprise text, alphanumerics, numbers, single words, phrases, short statements, long statements, expressions, syntax, source code statements, machine code, etc. Fine-tuning of a process 125A-n can comprise application of historical data 194, medical data 196, input data 108A-n, images 172A-n, text 176A-n, findings 179A-n, supplemental information 155A-n, summaries 151A-n, and suchlike, to the process 125A-n, the process 125A-n is correspondingly adjusted by application of the historical data 194, etc., such that, for example, weightings in the respective process 125A-n are adjusted by application of the historical data 194, and suchlike. As new information (e.g., input data 108A-n is processed) historical data 194 can be updated accordingly, and further, processes 125A-n fine-tuned.

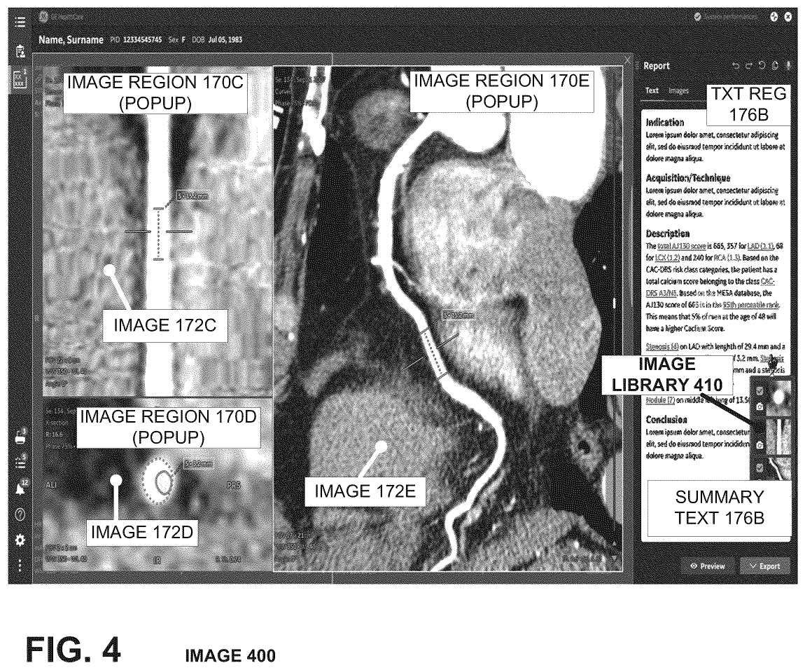

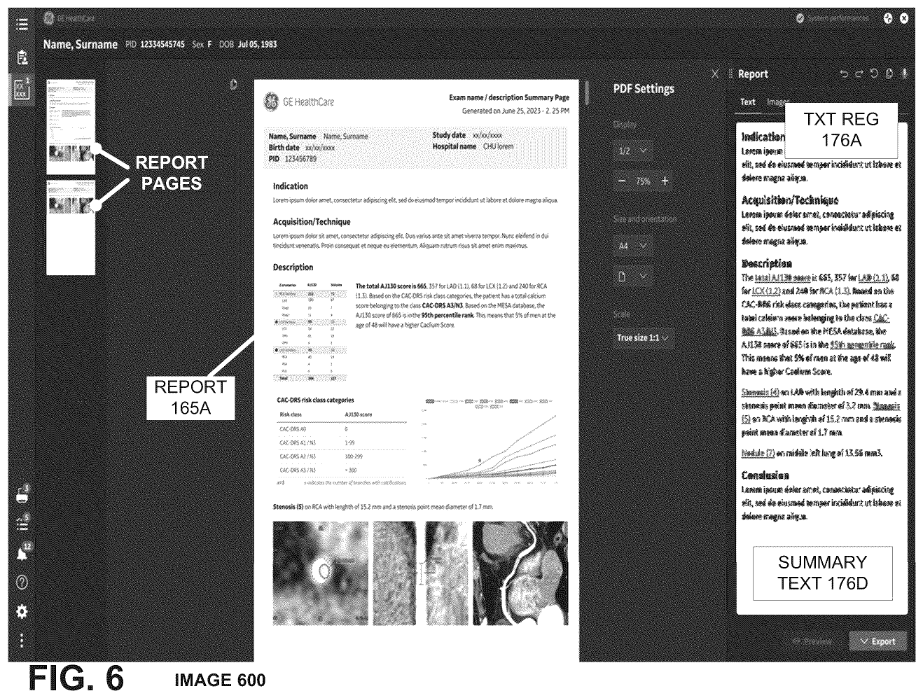

In accordance with one or more embodiments, and not as an exhaustive list, caregiver 103 can perform any of the following interactions with the summary screens 150A-n:
a) Caregiver 103 can select an exam presented in a worklist (e.g., one or more tasks to be performed regarding reviewing patient 102's medical condition 109A-n), wherein caregiver 103 can select the all data screen(s) 160A-n, and/or the summary screens 150A-n, as part of the review process.
b) Summary screens 150A-n can, at a minimum, be configured to present a single large viewport that provides one or more overviews of respective graphical objects/images 172A-n/findings 179A-n that have been automatically identified by any of the presentation component 115, summary component 120, and/or condition component 130, in conjunction with processes 125A-n.
c) Caregiver 103 can conduct various manipulations of the images 172A-n. For example, page image, zoom image, pan image, rotate image, change view type/render mode, and suchlike.
d) As previously mentioned, summary screens 150A-n can include a text region 174A-n (e.g., a text panel display portion) which can include text 176A-n, etc., providing a summary of the findings 179A-n. Respective portions of text 176A-n can be highlighted/linked to provide links 177A-n to supplemental information 155A-n. Per FIG. 1, while the text region 174A-n is presented as being located on the right side of summary screen 150A, text region 174A-n can be present on any region of the summary screen 150A. As previously mentioned, text presented in text region 174A-n can comprise text/information identified by the summary component 120. Caregiver 103 can select the respective links 177A-n in text 176A-n, whereupon selection of a respective link 177A-n, a popup (partial summary screen 150B) can be presented on the summary screen 150A, wherein the popup can be configured to be positioned on the summary screen 150A.
e) Caregiver 103 can “hover” a mouse cursor, or suchlike, over text 176A-n, whereby respective thumbnails of images 172A-n/107A-n can be presented that pertain to text 176A-n.
f) Caregiver 103 can select a link 177A-n to a process 125C dedicated to a specific aspect of the medical condition 109A-n of patient 102. For example, where the caregiver 103 is reviewing a summary of data, in findings 179A-n, regarding patient 102 suffering a cardiac condition 109C, the summary screen 150C can present respective aspects of the cardiac condition 109C, e.g., image 172C, findings 179A-n, and pertinent text 176C. However, a series of linked processes 125A-n can be available and selected by clicking respective links 177A-n. For example, process 125D may comprise algorithm(s)/application(s) configured to analyse one or more issues regarding a calcium score for patient 102, process 125E may comprise algorithm(s)/application(s) configured to analyse one or more issues regarding coronary analysis of patient 102's coronary system, process 125F may comprise algorithm(s)/application(s) configured to analyse one or more issues regarding stenosis of patient 102's coronary artery system, process 125G may comprise algorithm(s)/application(s) configured to analyse one or more issues regarding a fractional flow reserve (FFR) of patient 102's coronary region, and suchlike. As each process 125A-n is selected, information/findings 179A-n provided by the process/application can be presented on the summary screen 150A-n. Hence, while text 176A-n on summary screen 150A may summarize the respective analysis/findings 179A-n of processes 125A and 125B, a series of associated processes 125C-n are available for use by the caregiver 103 to further review the medical condition 109A-n of patient 102.
g) Text 176A-n presented on screen 150A-n/pop up, can be configured to be editable such that caregiver 103 can edit/annotate the text as required. Caregiver 103 can interact with summary screen 150A-n by any suitable means, each via a keyboard/interface (not shown) included in HMI 186, and suchlike, a microphone (not shown) incorporated into HMI 186, and suchlike, such that caregiver 103 can dictate text and/or instructions. Information presented on summary screen 150A-n (e.g., text 176A-n, images 172A-n, findings 179A-n, and suchlike) can be copy/pasted into a report 165A-n portion of the summary display 150A-n (as shown in FIG. 6).
h) Caregiver 103 can interact with the images 172A-n, such that by hovering/placing a cursor over the respective findings on image region 170A-n can cause corresponding text 176A-n in text region 174A-n to also be highlighted.
i) Caregiver 103 can further remove a finding 179A-n from the text region 174A-n, e.g., by right clicking a mouse/cursor on unwanted text 176A-n. By removing the finding 179A-n from the text region 174A-n, the finding 179A-n can also be removed from inclusion in the report 165A-n, e.g., preventing finding 179A-n/text 176A-n from being included in the exported report 165A-n.
j) Summary screen 150A-n can also include an image library tab (e.g., image library tab 410, per FIG. 4), which when selected, enables caregiver 103 to retrieve all/respective images 172A-n/images 107A-n/images available in historical data 194A-n and/or in medical data 196A-n to be automatically generated for presentation on the summary screen 150A-n. The respective images 172A-n, etc., can include any images that caregiver 103 has added to the collection of images 172A-n, e.g., using a copy to clipboard (screenshot) tool. Further, caregiver 103 can interact with the images 172A-n, etc., to facilitate image addition and/or image removal from the summary screen 150A-n, which can further lead to addition/removal of the respective image 172A-n, etc., from/to the report 165A-n. Further, the caregiver 103 can select an image 172A-n, etc., to copy/paste the image to the report 165A-n.
k) Summary screen 150A-n can further include a tab enabling a preview of the report 165A-n, e.g., prior to report 165A-n being selected, as well as an ability to review any document (e.g., medical report/paper) that may pertain to particular information, e.g., a finding 179A-n, presented on summary screen 150A-n.

As further shown, MDPS 110 can be communicatively coupled to a computer system 180. Computer system 180 can include a memory 184 that stores the respective computer executable components (e.g., presentation component 115, summary component 120, processes 125A-n, condition component 130, report component 135, confirmation component 140, vector component 710, similarity component 720, and suchlike) and further, a processor 182 configured to execute the computer executable components stored in the memory 184. Memory 184 can further be configured to store any of patient data 105A-n, medical data 106A-n, images 107A-n, input data 108A-n, historical data 194, medical data 196, images 172A-n, text 176A-n, summaries 151A-n, findings 179A-n, diagnoses 178A-n, reports 165A-n, supplemental information 155A-n, similarity indexes S₁₋ₙ, vectors Vn (e.g., similarity indexes 721A-n and vectors 711A-n, as further described herein), and suchlike. The computer system 180 can further include a human machine interface (HMI) 186 (e.g., a display, a graphical-user interface (GUI)) which can be configured to present various information including summary screens 150A-n, all data screens 160A-n, receive text/dictation instructions, mouse/cursor inputs, keyboard inputs, and suchlike. HMI 186 can include an interactive display/screen 187A-n to present the various summary screens 150A-n, all data screens 160A-n, reports 165A-n, supplemental information 155A-n, and suchlike. Computer system 180 can further include an I/O component 188 to receive and/or transmit historical data 194, medical data 196, input data 108A-n, findings 179A-n, diagnoses 178A-n, reports 165A-n, and suchlike. It is to be appreciated that while only one computer system 180 is presented in FIG. 1, multiple computer systems 180A-n can be utilized across system 100, such as a first computer system 180A is utilized in conjunction with MDPS 110, while a second computer system 180B is utilized by caregiver 103 to interact with MDPS 110 (e.g., via a second HMI 186A presenting summary screens 150A-n, all data screens 160A-n, reports 165A-n, supplemental information 155A-n, findings 179A-n, diagnoses 178A-n, and the like), and a third computer system 180C (e.g., an EMR system) is utilized to collect/generate input data 108A-n for submission to the MDPS 110.

Various communications 197A-n can be utilized across the system 100, between MDPS 110 (and included components), a system (not shown) facilitating entry/sourcing of input data 108A-n, computer system 180, etc. Communications 197A-n can include notifications (e.g., notification 750A-n, etc.), instructions, status updates, selections, data, information, interaction with any of the summary screen 150A-n, all data screen 160A-n, and information presented thereon, interaction with summary 151A-n, findings 179A-n, diagnosis 178A-n, links 177A-n, report 165A-n, input data 108A-n, confirmations 142A-n, and the like.

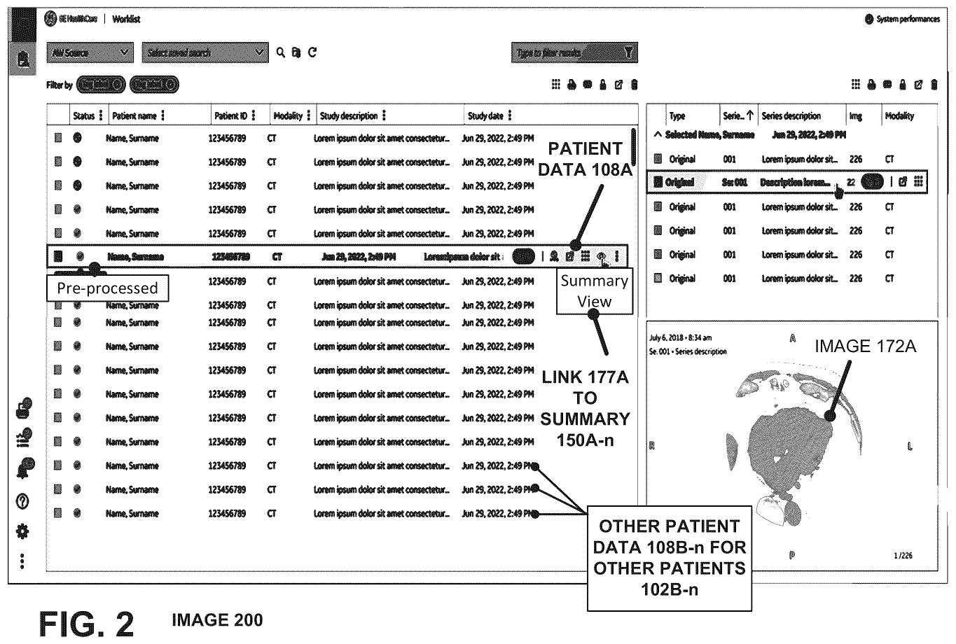

To provide further understanding of the various embodiments presented herein, FIGS. 2-6, images 200-600 present screen captures of a series of example screens, in accordance with one or more embodiments.

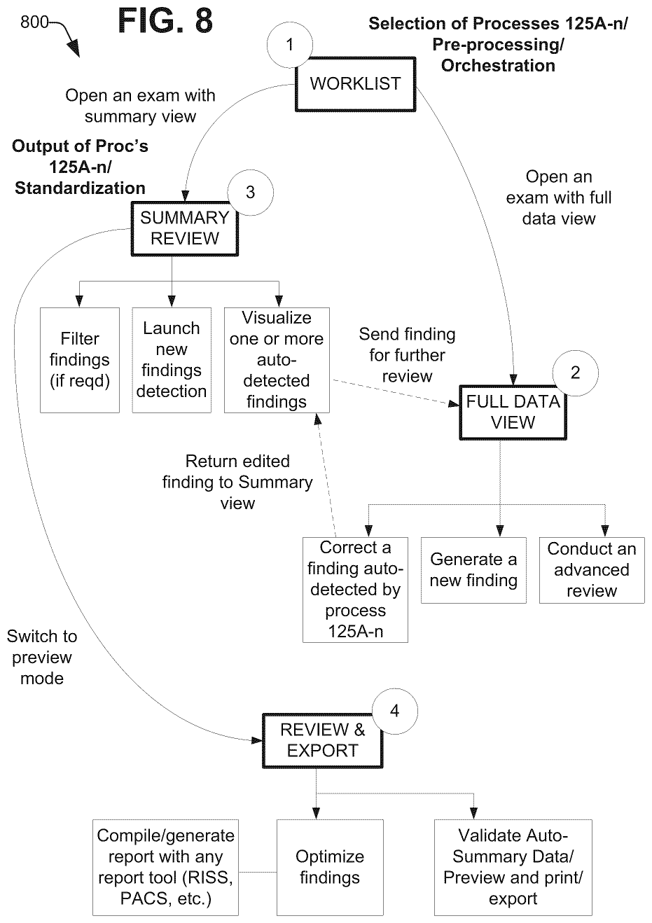

FIG. 2, image 200 presents a patient selection screen. In an embodiment, patient selection screen 200 (also referred to herein as a worklist, per FIG. 8) presents a list of respective patients 102A-n (and associated patient data 108A-n), and further a link 177A-n to present information pertaining to patient 102 (e.g., patient 102A) as a summary review (e.g., per summary screens 150A-n) rather than as an all data screen 160A-n. During selection of a record of patient 102, an image 172A pertaining to patient 102 can be presented on selection screen 200. Further, more than one medical condition 109A-n may pertain to patient 102, such that patient selection screen 200 can include a series of medical condition/diagnosis links 177A-n, which when selected can initiate implementation of respective processes 125A-n regarding the particular medical condition(s) 109A-n of patient 102.

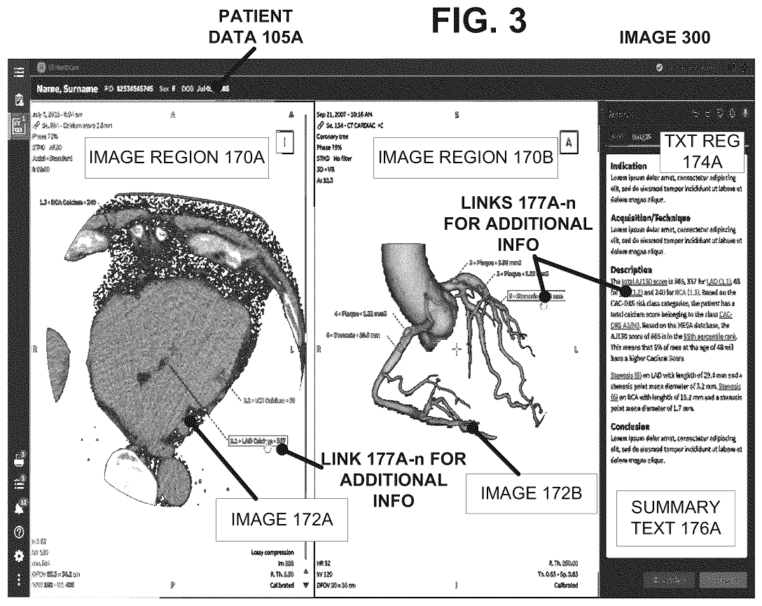

FIG. 3, image 300 presents a summary screen 150A, wherein summary screen 150A can be an initial summary screen generated by the summary component 120 in conjunction with processes 125A-n. Summary screen 150A can be presented in response to medical record of patient 102 being selected on selection screen 200. As shown, summary screen 150A can include patient data 105A, and further comprise a first screen region, image region 170A-n, wherein image region 170A-n can include various images 172A-n pertaining to the medical condition 109A-n of patient 102. For example, image region 170A includes image 172A, image region 170B include image 172B. The summary screen 150A can further comprise a second screen region, text region 174A-n, wherein text region 174A includes text 176A. Findings 179A-n can be presented on the summary screen 150A-n.

FIG. 4, image 400 presents a summary screen 150B, wherein summary screen 150B is an updated version of initial summary screen 150A, e.g., updated as a function of a hyperlink 177A-n being selected in text region 174A-n. New images 172C-E are presented in popup image regions 170C-E overlaying the originally presented image regions 170A-B and originally presented images 172A-B. As shown in FIG. 4, a selection of thumbnails are presented in updated text region 174B which when selected can cause images 172C-E to be presented.

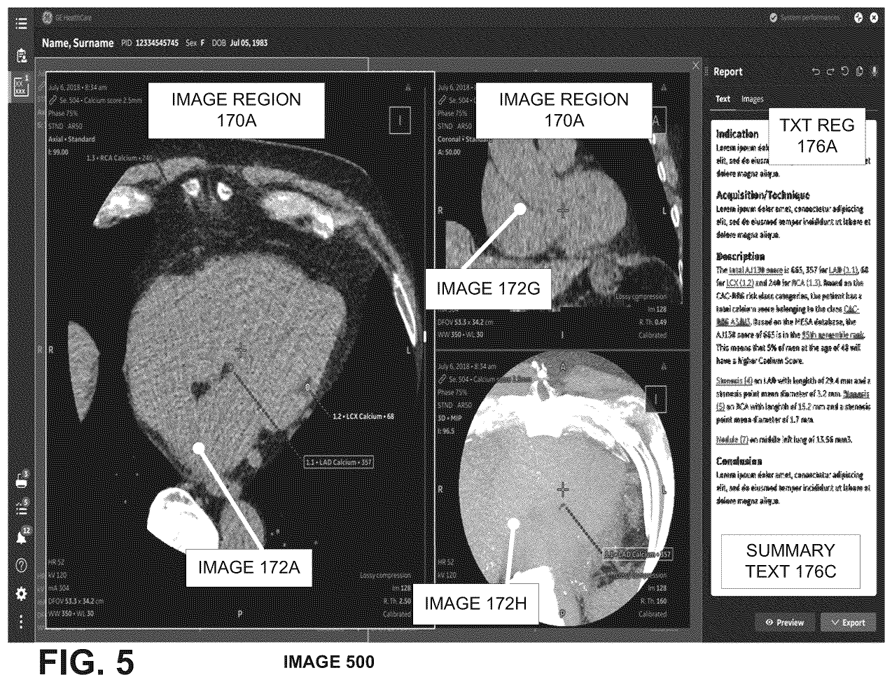

FIG. 5, image 500 presents a summary screen 150C, wherein summary screen 150C represents image region 170A/image 172A, while image region 170F presents images 172G and 172H. Images 172G and 172H can be presented in response to a link 177A-n being selected in an image (e.g., in image 172A), a link 177A-n in summary text 176C, and suchlike.

FIG. 6, image 600 presents a summary screen 150D, wherein summary screen 150D includes a rendition of report 165A and further, text region 174A. As previously mentioned, report 165A can be generated based on selection of a review complete tab, e.g., as detected by confirmation component 140. Respective pages of report 165A can be selected for review.

Returning to FIG. 1, condition component 130 (e.g., in conjunction with processes 125A-n) can be further configured to automatically identify one or more features in input data 108A-n, historical data 194, and/or medical data 196, wherein the term “one or more features” relates to any of a term, value, phrase, variable, parameter, criteria, image representation, medical condition 109A-n, medical reading, annotations (e.g., by caregiver 103 interacting with images 172A-n, findings 179A-n, and/or text 176A-n), image selection/removal (e.g., by caregiver 103 interacting with images 172A-n), and suchlike, pertaining to patient 102 that may be present in any of input data 108A-n, historical data 194, medical data 196.

Any suitable technology, methodology, and suchlike can be utilized to identify one or more features pertaining to a medical condition 109A-n of patient 102. In an aspect, at the time the input data 108A-n is received at MDPS 110, knowledge of the respective one or more features pertaining to patient 102 represented in input data 108A-n may be limited/unknown, e.g., by one or more caregivers 103A-n.

In an example embodiment, condition component 130 can be configured to compare a degree of similarity S between a feature in the input data 108A-n with the collection of features in historical data 194 and/or medical data 196 stored in memory 184. In an example embodiment, similarity S can range from a low degree of similarity (e.g., approaching 0 in a 0-1 similarity system indicating no match) through to a high degree of similarity (e.g., approaching 1.0 in a 0-1 similarity system indicating a match), and any intermediate degree of similarity therebetween. Measure of similarity can be based on/assessed/determined by any suitable/applicable criteria, e.g., substantially similar, S has a relative threshold level of similarity (e.g., S is compared to a threshold value T, equal to T, below T, above T), various levels of similarity, and the like.

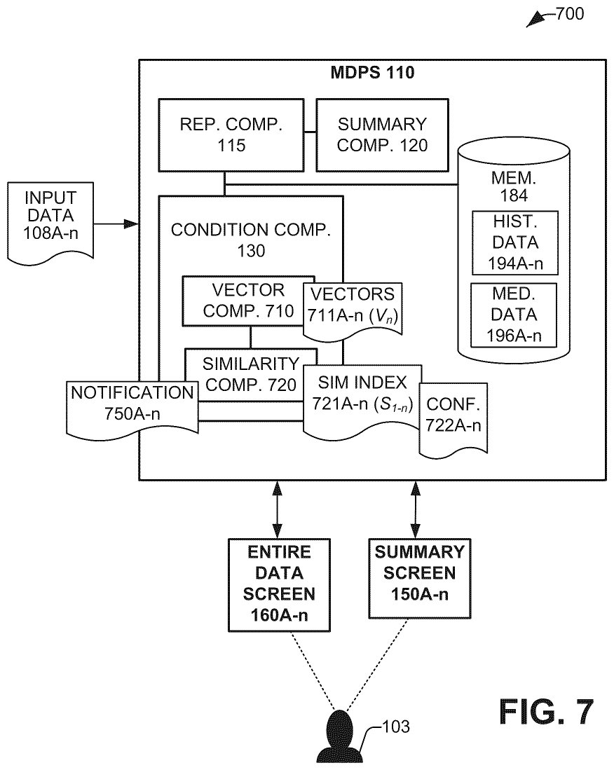

FIG. 7, system 700, further illustrates sub-components included in a condition component, in accordance with one or more embodiments. As shown in FIG. 7, condition component 130 can include a vector component 710 configured to process/vectorize the respective content/feature/element in input data 108A-n, historical data 194, medical data 196, etc. As part of processing the respective features of input data 108A-n, historical data 194, etc., each respective feature in each of input data 108A-n, historical data 194, etc., can be defined/represented by the vector component 710 as a vector V711A-n wherein the vector schema utilized can be any of a two-dimensional vector through to a multi-dimensional vector (e.g., a vector of many dimensions). The greater the similarity S between a first vector representation 711A (e.g., of first feature/content in input data 108A-n) and a second vector representation 711B (e.g., of second feature/content in historical data 194A-n, medical data 196A-n, and the like), the greater the confidence/inference 722A-n that the element represented by the first vector representation 711A relates to the element represented by the second vector 711B (e.g., the greater the similarity between vectors 711A and 711B the greater the likelihood of a medical condition 109A-n being identified correctly, the greater the level of confidence in a potential diagnosis 178A-n, the greater the likelihood that report 165A-n includes accurate information regarding a medical condition 109A-n of patient 102 and/or diagnosis 178A-n, and the like). Respective vectors V711A-n can be generated using any suitable approach, e.g., respective content/features can be expressed numerically, e.g., any of condition component 130, summary component 120, presentation component 115, and suchlike, e.g., in conjunction with one or more processes 125A-n can be configured to identify a feature/content, and the vector component 710 converts one or more portions of alphanumerics/text/numeric/symbols/content/annotations of the respective features/content in input data 108A-n, historical data 194, etc., into vectorized content.

Condition component 130 can further include a similarity component 720 configured to determine a degree of similarity S (e.g., a similarity index S₁₋ₙ) between vector representation V1 (e.g., vector 711A) of a feature in the input data 108A-n and a vector representation V2 (e.g., vector 711B) of feature in the historical data 194 and/or medical data 196 which have been previously identified/vectorized. For example, condition component 130 identifies the respective terms calcium score, stenosis data, coronary data, FFR, and suchlike in input data 108A-n and also the same, or potentially similar terms in historical data 194 and medical data 196. The respective terms can be vectorized by vector component 710, such that in an example of input data 108A has a parameter “calcium score” while historical data 194 has a parameter “calcium measurement”, calcium score can be represented as vector V1, while calcium measurement associated with a medical condition 109A-n of interest in either of historical data 194 and/or medical data 196 can be represented as vector V2. In the event of S indicates a high degree of confidence 722A-n in similarity that “calcium score” pertains to “calcium measurement”, an associated finding 179A-n can be presented, and if required, further review/analysis by condition component 130 can be performed regarding the medical condition 109A-n of patient 102 (e.g., is calcium score of patient 102 of concern?). For pertinent comparable parameters, the condition component 130 can be further configured to compare respective values/measurements for the particular parameter to determine whether patient 102 has a medical condition 109A-n. In the event of S indicates a low degree of confidence 722A-n in similarity that “calcium score” pertains to “calcium measurement”, further review of input data 108A-n, historical data 194, and medical data 196 can be performed to identify values, e.g., in a finding 179A-n, that pertain the parameter/medical condition 109A-n of concern. In an embodiment, the degree of confidence 722A-n relating to S, similarity 721A-n, can be presented on the summary screen 150A-n in conjunction with a diagnosis 178A-n to which the degree of confidence pertains. Accordingly, caregiver 103 can readily determine, and take into consideration, the degree of confidence with which condition component 130 identified any findings 179A-n and/or determined the diagnosis 178A-n for which the caregiver 103 approves.

Per FIG. 7, the condition component 130 can be further configured to generate a notification 750A-n indicating a status of diagnosing one or more medical conditions 109A-n of patient 102. Notification 750A-n can be applied to summary screen 150A-n, e.g., incorporated into text 176A-n, informing caregiver 103 of whether findings 179A-n indicate patient 102 has a particular medical condition 109A-n. The caregiver 103 can further review information presented, or available to be presented, on summary screen 150A-n to confirm/investigate the findings 179A-n of the one or more components included in MDPS 110. As previously mentioned, caregiver 103 can generate a confirmation of the diagnosis 178A-n via confirmation component 140.

FIG. 8, schematic 800, illustrates operational flow for diagnosis of a patient's medical condition, in accordance with one or more embodiments. The following comprises various operations 1-4 and various activities that can be respectively performed. It is to be appreciated that the numbering of operations 1-4 is arbitrary and any sequence of operations/activities can be performed as a caregiver 103 utilizes data/information presented on summary screen(s) 150A-n and full data screen(s) 160A-n.

At 800-1, an initial state of the diagnosis of patient 102 can be established. As previously mentioned, input data 108A-n can be available for patient 102 and received at MDPS 110. Per FIG. 2, input data 108A-n for patient 102 can be presented on patient selection screen 200, in conjunction with medical data for other patients. Upon selection of patient 102, respective processes 125A-n can be initiated (e.g., by presentation component 115, condition component 130, summary component 120, etc.) to initiate review of patient 102's input data 108A-n regarding one or more potential medical conditions 109A-n. Processes 125A-n can be utilized to review input data 108A-n to identify various anatomical markers in the input data 108A-n, thereby enabling implementation of respective processes 125A-n

At 800-2, in an example scenario of operation, caregiver 103 can select to review input data 108A-n and respective findings 179A-n, proposed diagnosis 178A-n, etc., via a full data screen 160A-n. As previously mentioned, full data screen 160A-n embodies a conventional approach to reviewing and diagnosing medical information (e.g., input data 108A-n) for patient 102. The medical information is available for caregiver 103 to review in what is effectively a step-through manner as the caregiver 103 reviews medical images, findings, and information, while attempting to diagnose patient 102's medical condition 109A-n.

At 800-3, per one or more embodiments presented herein, rather than presenting the entirety of input data 108A-n and any diagnoses, related data etc., one or more components included in MDPS 110 can be configured to present medical data pertaining to patient 102 in a summary form via summary screen 150A-n. As previously described, a summary component 120 in conjunction with processes 125A-n can be utilized to summarize the wealth of available information (e.g., in input data 108A-n, historical data 194, medical data 196, findings 179A-n, etc.). As shown in FIG. 3, summary screens 150A-n and full data screens 160A-n can interface such that while information is presented in a full rendition of full data screens 160A-n, the same information can be summarized on the summary screens 150A-n, as well as being presented in full via a pop-up window presented on summary screen 150A-n, and also temporary presentation of the full data screen 160A-n to edit the information there, before returning to the summary view. In the event of caregiver 103 wants to make an edit to the information, the edit (e.g., text, voice command, etc.) can be applied to summarized information on summary screen 150A, with the edit carried over to the entirety of information available for presentment on the full data screen 160A-n. Hence, while caregiver 103 may be interacting with summarized information (e.g., findings 179A-n), the MDPS 110 is configured to ensure the edits are captured. In another embodiment, while caregiver 103 may be interacting with summarized information on summary screen 150A, the entirety of the available information pertaining to the particular medical condition 109A-n can be provided on the summary screen 150A in a manner comparable to the information being presented on a full data screen 160A. Once caregiver 103 has conducted the required edits, the summary screen 150A can return to presenting a summary of the available information.

In another embodiment, while on the summary screen 150A-n, caregiver 103 can request a new analysis to be performed, e.g., which may not have been performed to date. The condition component 130 can implement respective processes 125A-n to enable the analysis to be performed, with the results (a) summarized on the summary screen 150A-n while a full set of information generated from the new analysis is available via the full data screen 160A-n.

At 800-4, as previously described, information (e.g., in input data 108A-n, historical data 194, medical data 196, findings 179A-n, proposed diagnosis 178A-n, etc.) can be reviewed by caregiver 103, and upon completion of the review, a report 165A-n can be generated. Report 165A-n can be generated for integration/use by any suitable technology/applications, e.g., rural integrated service system (RISS), picture archiving and communication system (PACS), and suchlike. Report 165A-n can be printed and/or exported to an external system.

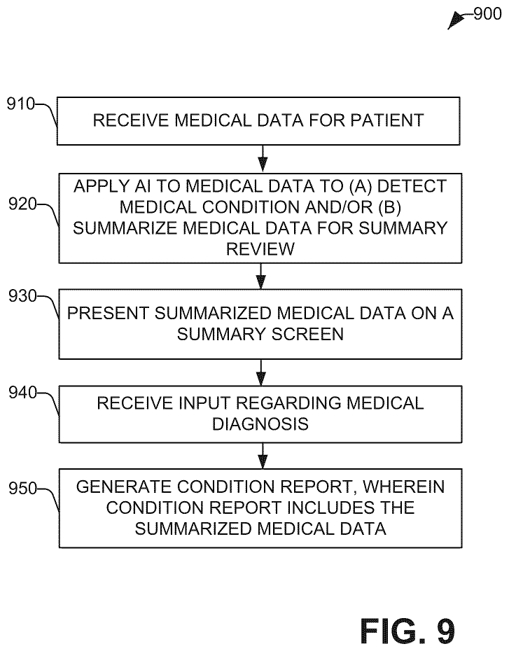

FIG. 9, via flowchart 900, presents a computer-implemented method for generating/presenting a summary of medical information for review, in accordance with an embodiment.

At 910, medical data (e.g., input data 108A-n) for a patient (e.g., patient 102) can be received at a medical data presentation system (MDPS) (e.g., MDPS 110).

At 920, various AI/ML techniques and technologies (e.g., processes 125A-n) can be applied (e.g., by presentation component 115, summary component 120, condition component 130) to the medical data to (a) identify one or more medical conditions (e.g., medical conditions 109A-n) which the patient may be suffering, and (b) to generate summary data (aka, first medical information, e.g., images 172A-n, text 176A-n, findings 179A-n). As previously mentioned, a plethora of medical information (aka, second medical information) is available (e.g., input data 108A-n, historical data 194A-n, medical data 196A-n) from which the medical condition(s) of the patient can be determined (e.g., in findings 179A-n) and a diagnosis (e.g., diagnosis 178A-n) derived. The various AI/ML techniques can be utilized to facilitate pre-processing of patient data (input data 108A-n) to automatically identify anatomical markers that can be utilized to identify historical data (e.g., historical data 194 and/or medical data 196) that pertain to the medical issue of concern (e.g., medical condition 109A-n) for patient 102. Identification of anatomical markers enables subsequent implementation of other processes (e.g., processes 125A-n) to determine/infer one or more medical conditions (e.g., medical condition 109A-n) pertaining to the patient (e.g., per findings 179A-n).

At 930, rather than presenting (e.g., on full data screen 160A-n) the second medical information for a medic (e.g., caregiver 103) to spend time reviewing to formulate a diagnosis, the first medical information comprising pertinent/important information (e.g., images 172A-n, text 176A-n, findings 179A-n) can be identified and presented on a summary screen (e.g., summary screen 150A). The AI/ML technologies can be further applied to either of the first medical data and/or the second medical data to automatically generate (e.g., by condition component 130) a finding/potential diagnosis (e.g., finding 179A-n/potential diagnosis 178A-n). The potential diagnosis can be presented on either of the summary screen or the full data screen, indicating (a) a degree of confidence in the automatically generated potential diagnosis and/or (b) whether the patient, has or has not, the medical condition (e.g., medical conditions 109A-n).

At 940, input can be received at the summary screen (e.g., via HMI 186) from the medic regarding whether the medic agrees with the diagnosis, or not. During review of information presented on the summary screen, the medic can access the second medical information in the event that medic requires information beyond that provided by the first medical information. The supplemental information (e.g., supplemental information 155A-n) can be presented on the summary screen in the form of pop-up windows, incorporated into existing text, supplement existing images, replace existing images and text, and suchlike. Depending upon the degree of interaction/amendment by the medic with the first medical information and/or second medical information, the edits, replacements, annotations, etc., can result in a third medical information being presented on the summary screen and/or the full data screen, whereby the third medical information reflects the updates, etc., the were performed by the medic. The diagnosis can be continually updated in accord with the medic's editing of the presented medical information.

At 950, a confirmation (e.g., confirmation 142A-n) can be received from the medic, whereby a report (e.g., report 165A-n) can be generated (e.g., by report component 135) for review (e.g., on a summary screen 150A-n). Medic can further edit the report as required, and when finished, can print/export the report.

As used herein, the terms “infer”, “inference”, “determine”, and suchlike, refer generally to the process of reasoning about or inferring states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources.

Per the various embodiments presented herein, various components included in MDPS 110, presentation component 115, condition component 130, summary component 120, and suchlike, can include AI/ML and reasoning techniques and technologies (e.g., processes 125A-n) that employ probabilistic and/or statistical-based analysis to prognose or infer an action that a user desires to be automatically performed. The various embodiments presented herein can utilize various machine learning-based schemes for carrying out various aspects thereof. For example, a process 125A-n (e.g., by presentation component 115, summary component 120, condition component 130, and suchlike) for determining content in the historical data 194/medical data 196 relating to content in the input data 108A-n (e.g., to determine a finding 179A-n), a process 125A-n (e.g., by presentation component 115, summary component 120, condition component 130, and suchlike) for determining information to be presented on a summary screen 150A-n, a process 125A-n (e.g., by presentation component 115, summary component 120, condition component 130, and suchlike) for automatically generating a potential diagnosis 178A-n based on the determined correlation(s) between the input data 108A-n and the historical data 194A-n/medical data 196A-n, and suchlike, as previously mentioned herein, can be facilitated via an automatic classifier system and process.

A classifier is a function that maps an input attribute vector, x=(x1, x2, x3, x4, xn), to a class label class(x). The classifier can also output a degree/measure of confidence that the input belongs to a class, that is, f(x)=confidence(class(x)), wherein the confidence can be based on the similarity 721A-n, e.g., low value for S indicates low confidence, high value for S indicates high confidence. Such classification can employ a probabilistic and/or statistical-based analysis (e.g., factoring into the analysis utilities and costs) to prognose or infer an action that a user desires to be automatically performed (e.g., identifying respective features presented in input data 108A-n and creation of information for summary screens 150A-n and diagnosis 178A-n, and operations related thereto).

A support vector machine (SVM) is an example of a classifier that can be employed. The SVM operates by finding a hypersurface in the space of possible inputs that splits the triggering input events from the non-triggering events in an optimal way. Intuitively, this makes the classification correct for testing data that is near, but not identical to training data. Other directed and undirected model classification approaches include, e.g., naïve Bayes, Bayesian networks, decision trees, neural networks, fuzzy logic models, and probabilistic classification models providing different patterns of independence can be employed. Classification as used herein is inclusive of statistical regression that is utilized to develop models of priority.

As will be readily appreciated from the subject specification, the various embodiments can employ classifiers that are explicitly trained (e.g., via a generic training data) as well as implicitly trained (e.g., via observing user behavior, receiving extrinsic information). For example, SVM's are configured via a learning or training phase within a classifier constructor and feature selection module. Thus, the classifier(s) can be used to automatically learn and perform a number of functions, including but not limited to determining according to predetermined criteria, content in input data 108A-n and historical data 194A-n/medical data 196A-n, and generate findings/diagnosis 178A-n based thereon, for example.

As described supra, inferences can be made, and automated operations performed, based on numerous pieces of information. For example, whether sufficient context is available to infer, with a high degree of confidence, a correlation between content in input data 108A-n and historical data 194A-n/medical data 196A-n, whether a finding/diagnosis 178A-n has been correctly applied to input data 108A-n, and suchlike, to enable a diagnosis to be made from images 172A-n and text 176A-n presented on summary screen 150A-n.

In review, the various embodiments presented herein enable:
a) selection of respective processes 125A-n to be utilized as a function of acquisition parameters (e.g., in input data 108A-n, applied by caregiver 103, and suchlike),
b) implementation of respective processes 125A-n facilitate pre-processing of input data 108A-n to automatically identify anatomical markers that can be utilized to identify historical data 194 and/or medical data 196 that pertain to the medical issue of concern for patient 102, and
c) implementation of respective processes 125A-n across the entirety of MDPS 110 to standardize presentation/rendering of medical information on summary screens 150A-n and full data screens 160A-n, as well as generation of reports 165A-n. E.g., one or more processes 125A-n can be utilized to communicate between different presentation systems how a finding is to be presented.

The various embodiments presented herein enable generation and operation of MDPS 110 which can be incorporated into a full medical ecosystem, e.g., from initial worklisting based on patient 102's medical condition 109A-n/symptoms/input data 108A-n, as well as providing capability to refine findings automatically generated by implementation of respective processes 125A-n, and further integrate MDPS 110 into external report tools (e.g., PACS, RISS) and medical services.

Further, the various embodiments presented herein enable a simplified interface (e.g., summary screens 150A-n) for caregiver 103 to review the automated findings generated by implementation of processes 125A-n, in a compact visual representation, while still providing for more detailed presentation either by the use of pop-ups and/or switching back and forth between the summarized data presented on summary screen 150A-n and the totality of data presented on full data screen 160A-n. Also, when implemented in a medical condition review ecosystem, MDPS 110 enables easy editing of medical data/findings/diagnosis, as well as implementation in research, review, report generation for local and external use, patient reports, etc. Further, more than one caregiver 103A-n can utilize the MDPS 110.

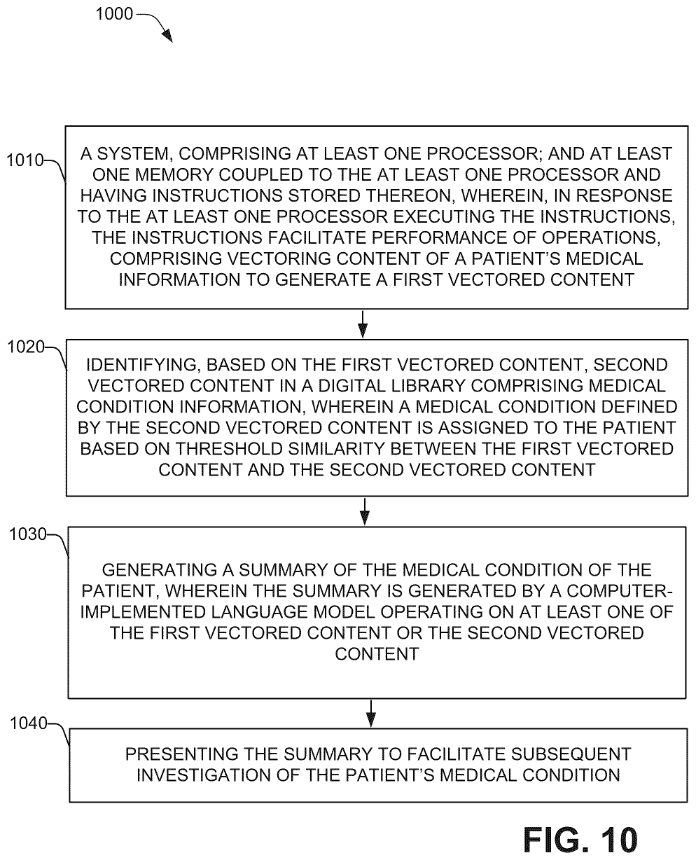

FIG. 10, via flowchart 1000, presents an example computer-implemented method for automatically and dynamically generating one or more summaries of patient information, further identify a potential medical condition for the patient, and further providing a potential diagnosis of the medical condition for subsequent review, in accordance with an embodiment.

At 1010, method 1000 can be performed by a system (e.g., MDPS 110), comprising at least one processor (e.g., processor 182A-n) and at least one memory (e.g., memory 184A-n) coupled to the at least one processor and having instructions stored thereon, wherein, in response to the at least one processor executing the instructions, the instructions facilitate performance of operations, comprising vectoring content of a patient's medical information (e.g., patient input data 108A-n) to generate a first vectored content (e.g., represented by a first vector 711A).

At 1020, method 1000 can further comprise identifying, based on the first vectored content, second vectored content (e.g., represented by a second vector 711B) in a digital library (e.g., historical data 194A-n, medical data 196A-n, supplemental information 155A-n, etc.) comprising medical condition information, wherein a medical condition (e.g., medical condition 109A-n) defined by the second vectored content is assigned to the patient based on threshold similarity between the first vectored content and the second vectored content.

At 1030, method 1000 can further comprise generating a summary (e.g., summary 151A-n including images 172A-n, text 176A-n) of the medical condition of the patient, wherein the summary is generated by a computer-implemented language model (e.g., process 125L) operating on at least one of the first vectored content or the second vectored content.

At 1040, method 1000 can further comprise presenting (e.g., on HMI 186) the summary to facilitate subsequent investigation (e.g., by caregiver 103) of the patient's medical condition.

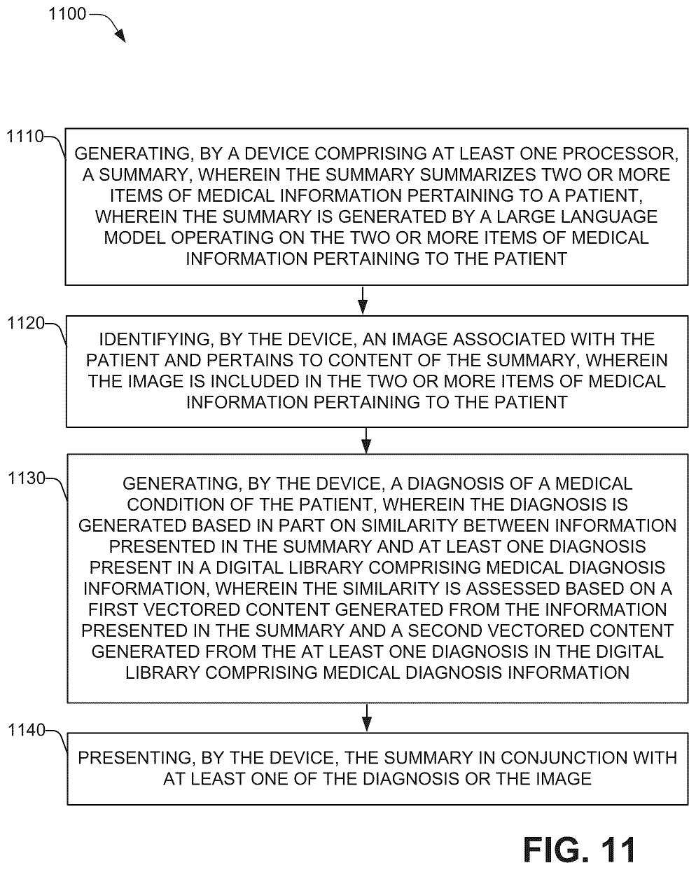

FIG. 11, via flowchart 1100, presents an example computer-implemented method for automatically and dynamically generating one or more summaries of patient information, further identify a potential medical condition for the patient, and further providing a potential diagnosis of the medical condition for subsequent review, in accordance with an embodiment.

At 1110, method 1100 can comprise generating, by a device (e.g., MDPS 110) comprising at least one processor (e.g., processor 182), a summary (e.g., summary 151A), wherein the summary summarizes two or more items (e.g., any of patient data 105A-n, medical data 106A-n, images 107A-n, input data 108A-n, a known medical condition 109A-n) of medical information pertaining to a patient (e.g., patient 102), wherein the summary is generated by a large language model (e.g., process 125A) operating on the two or more items of medical information pertaining to the patient.

At 1120, method 1100 can further comprise identifying, by the device, an image (e.g., any of images 107A-n) associated with the patient and pertains to content of the summary, wherein the image is included in the two or more items of medical information pertaining to the patient.

At 1130, method 1100 can further comprise generating, by the device, a diagnosis (e.g., diagnosis 178A-n) of a medical condition (e.g., medical condition 109A-n, finding 179A-n) of the patient, wherein the diagnosis is generated based in part on similarity (e.g., similarity S) between information presented in the summary and at least one diagnosis present in a digital library (e.g., memory 184) comprising medical diagnosis information (e.g., historical data 194A-n, medical data 196A-n, supplemental information 155A-n, etc.), wherein the similarity is assessed based on a first vectored content (e.g., first vectored content 711A) generated from the information presented in the summary and a second vectored content (e.g., second vectored content 711B) generated from the at least one diagnosis in the digital library comprising medical diagnosis information.

At 1140, method 1100 can further comprise presenting (e.g., on HMI 186), by the device, the summary in conjunction with at least one of the diagnosis or the image.

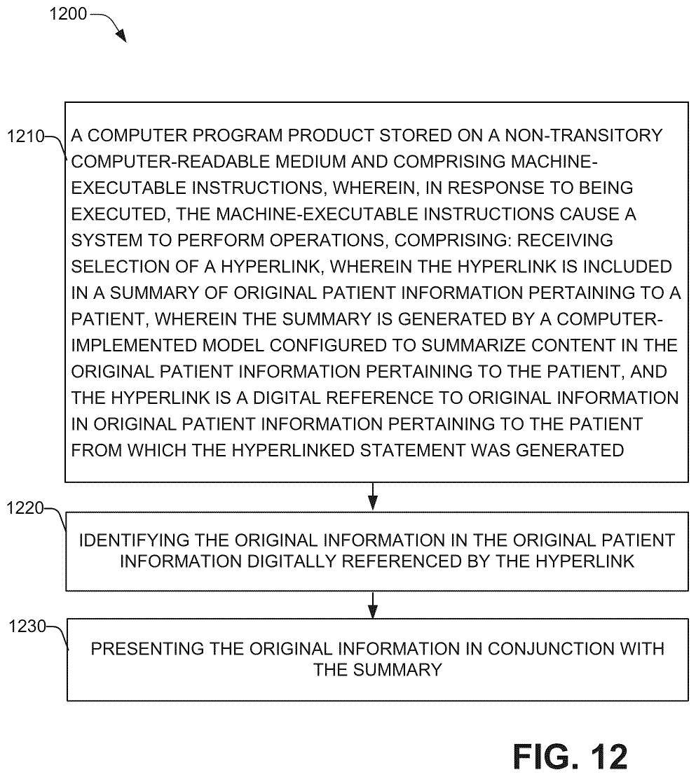

FIG. 12, via flowchart 1200, presents an example computer-implemented method for automatically and dynamically generating one or more summaries of patient information, further identify a potential medical condition for the patient, and further providing a potential diagnosis of the medical condition for subsequent review, in accordance with an embodiment.

At 1210, method 1200 can be performed with a computer program product stored on a non-transitory computer-readable medium (e.g., memory 184A-n) and comprising machine-executable instructions, wherein, in response to being executed (e.g., by processor 182A-n), the machine-executable instructions cause a system (e.g., MDPS 110) to perform operations, comprising receiving selection of a hyperlink (e.g., hyperlink 177A), wherein the hyperlink is included in a summary (e.g., summary 151A) of original patient information (e.g., any of patient data 105A-n, medical data 106A-n, images 107A-n, input data 108A-n, a known medical condition 109A-n) pertaining to a patient (e.g., patient 102), wherein the summary is generated by a computer-implemented model (e.g., process 125A) configured to summarize content in the original patient information pertaining to the patient, and the hyperlink is a digital reference to original information (e.g., content of any of patient data 105A-n, medical data 106A-n, images 107A-n, input data 108A-n, a known medical condition 109A-n) in original patient information pertaining to the patient from which the hyperlinked statement was generated.

At 1220, method 1200 can further comprise identifying the original information in the original patient information digitally referenced by the hyperlink.

At 1230, method 1200 can further comprise presenting (e.g., on HMI 186) the original information in conjunction with the summary.

### Example Applications and Use

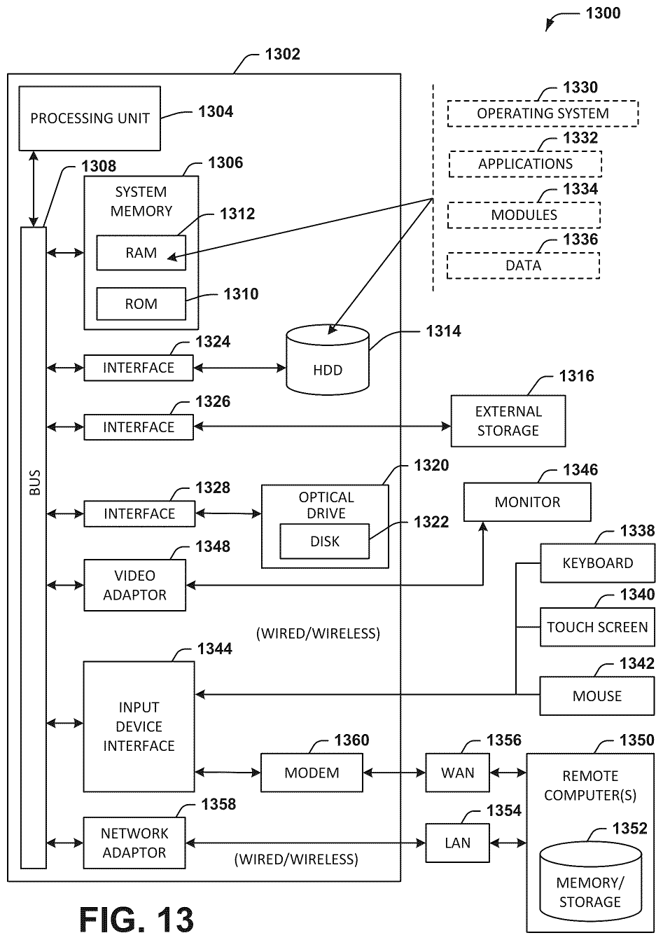

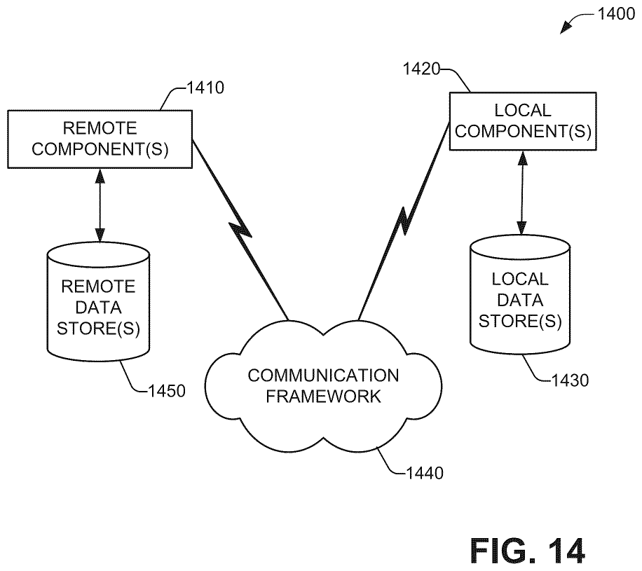

Turning next to FIGS. 13 and 14, a detailed description is provided of additional context for the one or more embodiments described herein with FIGS. 1-12.

In order to provide additional context for various embodiments described herein, FIG. 13 and the following discussion are intended to provide a brief, general description of a suitable computing environment 1300 in which the various embodiments described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the methods can be practiced with other computer system configurations, including single-processor or multiprocessor computer systems, minicomputers, mainframe computers, IoT devices, distributed computing systems, as well as personal computers, hand-held computing devices, microprocessor-based or programmable consumer electronics, and the like, each of which can be operatively coupled to one or more associated devices.

The embodiments illustrated herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 13, the example environment 1300 for implementing various embodiments of the aspects described herein includes a computer 1302, the computer 1302 including a processing unit 1304, a system memory 1306 and a system bus 1308. The system bus 1308 couples system components including, but not limited to, the system memory 1306 to the processing unit 1304. The processing unit 1304 can be any of various commercially available processors and may include a cache memory. Dual microprocessors and other multi-processor architectures can also be employed as the processing unit 1304.

The system bus 1308 can be any of several types of bus structure that can further interconnect to a memory bus (with or without a memory controller), a peripheral bus, and a local bus using any of a variety of commercially available bus architectures. The system memory 1306 includes ROM 1310 and RAM 1312. A basic input/output system (BIOS) can be stored in a non-volatile memory such as ROM, erasable programmable read only memory (EPROM), EEPROM, which BIOS contains the basic routines that help to transfer information between elements within the computer 1302, such as during startup. The RAM 1312 can also include a high-speed RAM such as static RAM for caching data.

The computer 1302 further includes an internal hard disk drive (HDD) 1314 (e.g., EIDE, SATA), one or more external storage devices 1316 (e.g., a magnetic floppy disk drive (FDD) 1316, a memory stick or flash drive reader, a memory card reader, etc.) and an optical disk drive 1320 (e.g., which can read or write from a CD-ROM disc, a DVD, a BD, etc.). While the internal HDD 1314 is illustrated as located within the computer 1302, the internal HDD 1314 can also be configured for external use in a suitable chassis (not shown). Additionally, while not shown in environment 1300, a solid-state drive (SSD) could be used in addition to, or in place of, an HDD 1314. The HDD 1314, external storage device(s) 1316 and optical disk drive 1322 can be connected to the system bus 1308 by an HDD interface 1324, an external storage interface 1326 and an optical drive interface 1328, respectively. The interface 1324 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1302, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1312, including an operating system 1330, one or more application programs 1332, other program modules 1334 and program data 1336. All or portions of the operating system, applications, modules, and/or data can also be cached in the RAM 1312. The systems and methods described herein can be implemented utilizing various commercially available operating systems or combinations of operating systems.

Computer 1302 can optionally comprise emulation technologies. For example, a hypervisor (not shown) or other intermediary can emulate a hardware environment for operating system 1330, and the emulated hardware can optionally be different from the hardware illustrated in FIG. 13. In such an embodiment, operating system 1330 can comprise one virtual machine (VM) of multiple VMs hosted at computer 1302. Furthermore, operating system 1330 can provide runtime environments, such as the Java runtime environment or the .NET framework, for applications 1332. Runtime environments are consistent execution environments that allow applications 1332 to run on any operating system that includes the runtime environment. Similarly, operating system 1330 can support containers, and applications 1332 can be in the form of containers, which are lightweight, standalone, executable packages of software that include, e.g., code, runtime, system tools, system libraries and settings for an application.

Further, computer 1302 can comprise a security module, such as a trusted processing module (TPM). For instance with a TPM, boot components hash next in time boot components, and wait for a match of results to secured values, before loading a next boot component. This process can take place at any layer in the code execution stack of computer 1302, e.g., applied at the application execution level or at the operating system (OS) kernel level, thereby enabling security at any level of code execution.

A user can enter commands and information into the computer 1302 through one or more wired/wireless input devices, e.g., a keyboard 1338, a touch screen 1340, and a pointing device, such as a mouse 1342. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1304 through an input device interface 1344 that can be coupled to the system bus 1308, but can be connected by other interfaces, such as a parallel port, an IEEE 1394 serial port, a game port, a USB port, an IR interface, a BLUETOOTH® interface, etc.

A monitor 1346 or other type of display device can be also connected to the system bus 1308 via an interface, such as a video adapter 1348. In addition to the monitor 1346, a computer typically includes other peripheral output devices (not shown), such as speakers, printers, etc.

The computer 1302 can operate in a networked environment using logical connections via wired and/or wireless communications to one or more remote computers, such as a remote computer(s) 1350. The remote computer(s) 1350 can be a workstation, a server computer, a router, a personal computer, portable computer, microprocessor-based entertainment appliance, a peer device or other common network node, and typically includes many or all of the elements described relative to the computer 1302, although, for purposes of brevity, only a memory/storage device 1352 is illustrated. The logical connections depicted include wired/wireless connectivity to a local area network (LAN) 1354 and/or larger networks, e.g., a wide area network (WAN) 1356. Such LAN and WAN networking environments are commonplace in offices and companies, and facilitate enterprise-wide computer networks, such as intranets, all of which can connect to a global communications network, e.g., the internet.

When used in a LAN networking environment, the computer 1302 can be connected to the local network 1354 through a wired and/or wireless communication network interface or adapter 1358. The adapter 1358 can facilitate wired or wireless communication to the LAN 1354, which can also include a wireless access point (AP) disposed thereon for communicating with the adapter 1358 in a wireless mode.

When used in a WAN networking environment, the computer 1302 can include a modem 1360 or can be connected to a communications server on the WAN 1356 via other means for establishing communications over the WAN 1356, such as by way of the internet. The modem 1360, which can be internal or external and a wired or wireless device, can be connected to the system bus 1308 via the input device interface 1344. In a networked environment, program modules depicted relative to the computer 1302 or portions thereof, can be stored in the remote memory/storage device 1352. It will be appreciated that the network connections shown are example and other means of establishing a communications link between the computers can be used.

When used in either a LAN or WAN networking environment, the computer 1302 can access cloud storage systems or other network-based storage systems in addition to, or in place of, external storage devices 1316 as described above. Generally, a connection between the computer 1302 and a cloud storage system can be established over a LAN 1354 or WAN 1356 e.g., by the adapter 1358 or modem 1360, respectively. Upon connecting the computer 1302 to an associated cloud storage system, the external storage interface 1326 can, with the aid of the adapter 1358 and/or modem 1360, manage storage provided by the cloud storage system as it would other types of external storage. For instance, the external storage interface 1326 can be configured to provide access to cloud storage sources as if those sources were physically connected to the computer 1302.

The computer 1302 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

The above description includes non-limiting examples of the various embodiments. It is, of course, not possible to describe every conceivable combination of components or methodologies for purposes of describing the disclosed subject matter, and one skilled in the art may recognize that further combinations and permutations of the various embodiments are possible. The disclosed subject matter is intended to embrace all such alterations, modifications, and variations that fall within the spirit and scope of the appended claims.

Referring now to details of one or more elements illustrated at FIG. 14, an illustrative cloud computing environment 1400 is depicted. FIG. 14 is a schematic block diagram of a computing environment 1400 with which the disclosed subject matter can interact. The system 1400 comprises one or more remote component(s) 1410. The remote component(s) 1410 can be hardware and/or software (e.g., threads, processes, computing devices). In some embodiments, remote component(s) 1410 can be a distributed computer system, connected to a local automatic scaling component and/or programs that use the resources of a distributed computer system, via communication framework 1440. Communication framework 1440 can comprise wired network devices, wireless network devices, mobile devices, wearable devices, radio access network devices, gateway devices, femtocell devices, servers, etc.

The system 1400 also comprises one or more local component(s) 1420. The local component(s) 1420 can be hardware and/or software (e.g., threads, processes, computing devices). In some embodiments, local component(s) 1420 can comprise an automatic scaling component and/or programs that communicate/use the remote resources 1410 and 1420, etc., connected to a remotely located distributed computing system via communication framework 1440.

One possible communication between a remote component(s) 1410 and a local component(s) 1420 can be in the form of a data packet adapted to be transmitted between two or more computer processes. Another possible communication between a remote component(s) 1410 and a local component(s) 1420 can be in the form of circuit-switched data adapted to be transmitted between two or more computer processes in radio time slots. The system 1400 comprises a communication framework 1440 that can be employed to facilitate communications between the remote component(s) 1410 and the local component(s) 1420, and can comprise an air interface, e.g., Uu interface of a UMTS network, via a long-term evolution (LTE) network, etc. Remote component(s) 1410 can be operably connected to one or more remote data store(s) 1450, such as a hard drive, solid state drive, SIM card, device memory, etc., that can be employed to store information on the remote component(s) 1410 side of communication framework 1440. Similarly, local component(s) 1420 can be operably connected to one or more local data store(s) 1430, that can be employed to store information on the local component(s) 1420 side of communication framework 1440.

With regard to the various functions performed by the above described components, devices, circuits, systems, etc., the terms (including a reference to a “means”) used to describe such components are intended to also include, unless otherwise indicated, any structure(s) which performs the specified function of the described component (e.g., a functional equivalent), even if not structurally equivalent to the disclosed structure. In addition, while a particular feature of the disclosed subject matter may have been disclosed with respect to only one of several implementations, such feature may be combined with one or more other features of the other implementations as may be desired and advantageous for any given or particular application.

The terms “exemplary” and/or “demonstrative” as used herein are intended to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent structures and techniques known to one skilled in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

The term “or” as used herein is intended to mean an inclusive “or” rather than an exclusive “or.” For example, the phrase “A or B” is intended to include instances of A, B, and both A and B. Additionally, the articles “a” and “an” as used in this application and the appended claims should generally be construed to mean “one or more” unless either otherwise specified or clear from the context to be directed to a singular form.

The term “set” as employed herein excludes the empty set, i.e., the set with no elements therein. Thus, a “set” in the subject disclosure includes one or more elements or entities. Likewise, the term “group” as utilized herein refers to a collection of one or more entities.

The terms “first,” “second,” “third,” and so forth, as used in the claims, unless otherwise clear by context, is for clarity only and doesn't otherwise indicate or imply any order in time. For instance, “a first determination,” “a second determination,” and “a third determination,” does not indicate or imply that the first determination is to be made before the second determination, or vice versa, etc.

As used in this disclosure, in some embodiments, the terms “component,” “system” and the like are intended to refer to, or comprise, a computer-related entity or an entity related to an operational apparatus with one or more specific functionalities, wherein the entity can be either hardware, a combination of hardware and software, software, or software in execution. As an example, a component can be, but is not limited to being, a process running on a processor, a processor, an object, an executable, a thread of execution, computer-executable instructions, a program, and/or a computer. By way of illustration and not limitation, both an application running on a server and the server can be a component.

One or more components can reside within a process and/or thread of execution and a component can be localized on one computer and/or distributed between two or more computers. In addition, these components can execute from various computer readable media having various data structures stored thereon. The components can communicate via local and/or remote processes such as in accordance with a signal having one or more data packets (e.g., data from one component interacting with another component in a local system, distributed system, and/or across a network such as the internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software or firmware that confers at least in part the functionality of the electronic components. While various components have been illustrated as separate components, it will be appreciated that multiple components can be implemented as a single component, or a single component can be implemented as multiple components, without departing from example embodiments.

The term “facilitate” as used herein is in the context of a system, device or component “facilitating” one or more actions or operations, in respect of the nature of complex computing environments in which multiple components and/or multiple devices can be involved in some computing operations. Non-limiting examples of actions that may or may not involve multiple components and/or multiple devices comprise transmitting or receiving data, establishing a connection between devices, determining intermediate results toward obtaining a result, etc. In this regard, a computing device or component can facilitate an operation by playing any part in accomplishing the operation. When operations of a component are described herein, it is thus to be understood that where the operations are described as facilitated by the component, the operations can be optionally completed with the cooperation of one or more other computing devices or components, such as, but not limited to, sensors, antennae, audio and/or visual output devices, other devices, etc.

Further, the various embodiments can be implemented as a method, apparatus or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable (or machine-readable) device or computer-readable (or machine-readable) storage/communications media. For example, computer readable storage media can comprise, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

Moreover, terms such as “mobile device equipment,” “mobile station,” “mobile,” “subscriber station,” “access terminal,” “terminal,” “handset,” “communication device,” “mobile device” (and/or terms representing similar terminology) can refer to a wireless device utilized by a subscriber or mobile device of a wireless communication service to receive or convey data, control, voice, video, sound, gaming or substantially any data-stream or signaling-stream. The foregoing terms are utilized interchangeably herein and with reference to the related drawings. Likewise, the terms “access point (AP),” “Base Station (BS),” “BS transceiver,” “BS device,” “cell site,” “cell site device,” “gNode B (gNB),” “evolved Node B (eNode B, eNB),” “home Node B (HNB)” and the like, refer to wireless network components or appliances that transmit and/or receive data, control, voice, video, sound, gaming or substantially any data-stream or signaling-stream from one or more subscriber stations. Data and signaling streams can be packetized or frame-based flows.

Furthermore, the terms “device,” “communication device,” “mobile device,” “subscriber,” “client entity,” “consumer,” “client entity,” “entity” and the like are employed interchangeably throughout, unless context warrants particular distinctions among the terms. It should be appreciated that such terms can refer to human entities or automated components supported through artificial intelligence (e.g., a capacity to make inference based on complex mathematical formalisms), which can provide simulated vision, sound recognition and so forth.

It should be noted that although various aspects and embodiments are described herein in the context of 5G or other next generation networks, the disclosed aspects are not limited to a 5G implementation, and can be applied in other network next generation implementations, such as sixth generation (6G), or other wireless systems. In this regard, aspects or features of the disclosed embodiments can be exploited in substantially any wireless communication technology. Such wireless communication technologies can include universal mobile telecommunications system (UMTS), global system for mobile communication (GSM), code division multiple access (CDMA), wideband CDMA (WCMDA), CDMA2000, time division multiple access (TDMA), frequency division multiple access (FDMA), multi-carrier CDMA (MC-CDMA), single-carrier CDMA (SC-CDMA), single-carrier FDMA (SC-FDMA), orthogonal frequency division multiplexing (OFDM), discrete Fourier transform spread OFDM (DFT-spread OFDM), filter bank based multi-carrier (FBMC), zero tail DFT-spread-OFDM (ZT DFT-s-OFDM), generalized frequency division multiplexing (GFDM), fixed mobile convergence (FMC), universal fixed mobile convergence (UFMC), unique word OFDM (UW-OFDM), unique word DFT-spread OFDM (UW DFT-Spread-OFDM), cyclic prefix OFDM (CP-OFDM), resource-block-filtered OFDM, wireless fidelity (Wi-Fi), worldwide interoperability for microwave access (WiMAX), wireless local area network (WLAN), general packet radio service (GPRS), enhanced GPRS, third generation partnership project (3GPP), long term evolution (LTE), 5G, third generation partnership project 2 (3GPP2), ultra-mobile broadband (UMB), high speed packet access (HSPA), evolved high speed packet access (HSPA+), high-speed downlink packet access (HSDPA), high-speed uplink packet access (HSUPA), Zigbee, or another institute of electrical and electronics engineers (IEEE) 802.12 technology.

It is to be understood that when an element is referred to as being “coupled” to another element, it can describe one or more different types of coupling including, but not limited to, chemical coupling, communicative coupling, electrical coupling, electromagnetic coupling, operative coupling, optical coupling, physical coupling, thermal coupling, and/or another type of coupling. Likewise, it is to be understood that when an element is referred to as being “connected” to another element, it can describe one or more different types of connecting including, but not limited to, electrical connecting, electromagnetic connecting, operative connecting, optical connecting, physical connecting, thermal connecting, and/or another type of connecting.

The description of illustrated embodiments of the subject disclosure as provided herein, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as one skilled in the art can recognize. In this regard, while the subject matter has been described herein in connection with various embodiments and corresponding drawings, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

What is claimed is:

1. A system, comprising:
at least one processor; and
at least one memory coupled to the at least one processor and having instructions stored thereon, wherein, in response to the at least one processor executing the instructions, the instructions facilitate performance of operations, comprising:
vectoring content of a patient's medical information to generate a first vectored content;
identifying, based on the first vectored content, second vectored content in a digital library comprising medical condition information, wherein a medical condition defined by the second vectored content is assigned to the patient based on threshold similarity between the first vectored content and the second vectored content;
generating a summary of the medical condition of the patient, wherein the summary is generated by a computer-implemented language model operating on at least one of the first vectored content or the second vectored content; and
presenting the summary to facilitate subsequent investigation of the patient's medical condition.

2. The system of claim 1, wherein the summary includes a hyperlinked statement, wherein the hyperlinked statement is a digital reference to information in the patient's medical information from which the hyperlinked statement was generated, wherein the operations further comprise:
detecting selection of the hyperlinked statement; and
presenting the patient's medical information from which the hyperlinked statement was derived.

3. The system of claim 1, wherein the patient's medical information comprises at least one of personal data pertaining to the patient, medical data pertaining to the patient, an image of the patient, or an image of at least one organ of the patient.

4. The system of claim 1, wherein the operations further comprise:
determining, from the patient's medical information, a diagnosis of the patient's medical condition, wherein the diagnosis comprises generating third vectored content from the patient's medical information and identifying a fourth vectored content in the digital library comprising the medical condition information being similar to the third vectored content; and
presenting the diagnosis for further investigation of the patient's medical condition.

5. The system of claim 4, wherein the operations further comprise:
determining a degree of confidence in the appropriateness of the diagnosis to the patient's medical condition, wherein the degree of confidence is based on a measure of similarity between the third vectored content and the fourth vectored content; and
presenting the degree of confidence with the diagnosis.

6. The system of claim 4, wherein the operations further comprise:
receiving a confirmation of the diagnosis; and
in response to receiving the confirmation of the diagnosis, generating a report presenting a summary of the patient's medical condition and the diagnosis.

7. The system of claim 1, wherein the patient's medical information is first medical information and the summary of the medical information is a summary of the first medical information, wherein the operations further comprise:
detecting an edit applied to the summary of the first medical information, wherein the edit comprises addition or removal of information from the summary of the first medical information.

8. The system of claim 7, wherein the operations further comprise:
updating the first medical information to generate second medical information, wherein the second medical information comprises the edit applied to the summary of the first medical information.

9. A computer-implemented method, comprising:
generating, by a device comprising at least one processor, a summary, wherein the summary summarizes two or more items of medical information pertaining to a patient, wherein the summary is generated by a large language model operating on the two or more items of medical information pertaining to the patient;
identifying, by the device, an image associated with the patient and pertains to content of the summary, wherein the image is included in the two or more items of medical information pertaining to the patient;
generating, by the device, a diagnosis of a medical condition of the patient, wherein the diagnosis is generated based in part on similarity between information presented in the summary and at least one diagnosis present in a digital library comprising medical diagnosis information, wherein the similarity is assessed based on a first vectored content generated from the information presented in the summary and a second vectored content generated from the at least one diagnosis in the digital library comprising medical diagnosis information; and
presenting, by the device, the summary in conjunction with at least one of the diagnosis or the image.

10. The computer-implemented method of claim 9, wherein the summary includes a hyperlink to original information sourced from the two or more items of medical information from which the summary was generated, wherein the computer-implemented method further comprising:
detecting, by the device, selection of the hyperlink; and
in response to detecting, by the device, selection of the hyperlink, presenting, by the device, the original information in conjunction with the summary in conjunction with at least one of the diagnosis or the image.

11. The computer-implemented method of claim 9, wherein the hyperlink in the summary is a first hyperlink to first information sourced from the two or more items of medical information which the summary was generated, and wherein the medical image further comprises a second hyperlink pertaining to second information available in the two or more items of medical information.

12. The computer-implemented method of claim 9, further comprising:
detecting, by the device, an edit to the summary; and
updating, by the device, information in the original information in accordance with the edit to the summary.

13. The computer-implemented method of claim 11, wherein the edit to the summary comprises addition, modification, or removal of information from the summary.

14. The computer-implemented method of claim 12, further comprising:
updating, by the device, the original information to generate second information, wherein the second information comprises the edit applied to the summary of the original information.

15. The computer-implemented method of claim 9, wherein the original information comprises at least one of a medical image or medical condition information.

16. The computer-implemented method of claim 15, further comprising:
generating, by the device, a degree of confidence in the applicability of the diagnosis to the patient medical condition, wherein the degree of confidence is based on a measure of similarity between the first vectored content generated from the information presented in the summary and the second vectored content generated from the at least one diagnosis in the digital library comprising medical diagnosis information; and
presenting, by the device, the degree of confidence in conjunction with the diagnosis.

17. A computer program product stored on a non-transitory computer-readable medium and comprising machine-executable instructions, wherein, in response to being executed, the machine-executable instructions cause a system to perform operations, comprising:
receiving selection of a hyperlink, wherein the hyperlink is included in a summary of original patient information pertaining to a patient, wherein the summary is generated by a computer-implemented model configured to summarize content in the original patient information pertaining to the patient, and the hyperlink is a digital reference to original information in original patient information pertaining to the patient from which the hyperlinked statement was generated;
identifying the original information in the original patient information digitally referenced by the hyperlink; and
presenting the original information in conjunction with the summary.

18. The computer program product according to claim 17, wherein the hyperlink is a first hyperlink, wherein the original patient information pertaining to the patient is first patient information utilized to create the summary, the operations further comprising:
receiving selection of a second hyperlink, wherein the second hyperlink is included in an image presented in conjunction with the summary, and the second hyperlink links to second patient information pertaining to the patient;
identifying the second patient information pertaining to the hyperlink; and
presenting the second patient information.

19. The computer program product according to claim 17, the operations further comprising:
comparing the first patient information and the second patient information with medical condition information, wherein the medical condition information is stored in a digital library comprising medical diagnosis information and includes information pertaining to a medical condition;
identifying, in the medical condition information, the medical condition, wherein identification of the medical condition information comprises comparing similarity of at least one of first vectored content generated from the first patient information or second vectored content generated from the second patient information with third vectored content generated from the medical condition information stored in the digital library comprising medical diagnosis information;
generating a diagnosis of the medical condition, wherein the diagnosis is generated based on a comparison of at least one of the first patient information or the second patient information with diagnosis information included in the digital library comprising medical diagnosis information, wherein the diagnosis is based on measure of similarity between at least one of the first vectored content or the second vectored content with a third vectored content generated for the diagnosis; and
presenting the diagnosis for review.

20. The computer program product according to claim 19, the operations further comprising:
generating a degree of confidence for the diagnosis, wherein the degree of confidence is based on at least one of a first relatedness measure of the first vectored content to the medical condition or a second relatedness measure of the second vectored content to the medical condition; and
presenting the degree of confidence in conjunction with the diagnosis.
